(19) United States
(12) Patent Application Publication
(10) Pub. No.: US 2025/0371704 A1
(43) Pub. Date: Dec. 4, 2025

(54) MACHINE LEARNING FRAMEWORK FOR BREAST CANCER HISTOLOGIC GRADING

(71) Applicant: Verily Life Sciences LLC, Dallas, TX (US)

(72) Inventors: Ronnachai Jaroensri, Dallas, TX (US); Fraser Tan, Dallas, TX (US); Yun Liu, Dallas, TX (US); Kunal Nagpal, Dallas, TX (US); Ellery Wulczyn, Dallas, TX (US); Craig Mermel, Dallas, TX (US); Dave Steiner, Dallas, TX (US); Po-Hsuan Cameron Chen, Dallas, TX (US)

(21) Appl. No.: 19/110,184

(22) PCT Filed: Oct. 3, 2023
(86) PCT No.: PCT/US2023/075861

§ 371 Date: Mar. 10, 2025

Related U.S. Application Data
(60) Provisional application No. 63/413,173, filed on Oct. 4, 2022.

(51) Int. Cl. G06T 7/00; A61B 10/00; G06N 3/045; G06N 3/0464; G06T 7/11; G06V 10/26; G06V 10/764; G06V 10/82; G06V 20/69

(52) U.S. Cl. CPC G06T 7/0012; G06N 3/045; G06N 3/0464; G06T 7/11; G06V 10/26; G06V 10/764; G06V 10/82; G06V 20/695; G06V 20/698; A61B 10/0041; G06T 2207/20081; G06T 2207/20084; G06T 2207/30024; G06T 2207/30068; G06T 2207/30096; G06T 2207/30242; G06V 2201/03

(57) ABSTRACT

A machine learning framework for breast cancer histologic grading is described herein. In an example, a method involves accessing a whole slide image of a specimen. The image is processed using a first, second, third, and fourth machine learning process. A first output of the first machine learning process indicates portions of the image predicted to depict tumor cells. A second output of the second machine learning process corresponds to a mitotic count predicted score for a mitotic count depicted in the image, a third output of the third machine learning process corresponds to a nuclear pleomorphism predicted score for nuclear pleomorphism depicted in the image, and a fourth output of the fourth machine learning process corresponds to a tubule formation predicted score for tubule formation depicted in the image. A combined score of a predicted histologic grade of a disease in the image is generated based on the outputs.

### CROSS-REFERENCES TO RELATED APPLICATIONS

This international application claims priority to U.S. Patent Application No. 63/413,173, filed on Oct. 4, 2022, the disclosure of which is herein incorporated by reference in its entirety for all purposes.

### BACKGROUND OF THE INVENTION

The present disclosure relates to digital pathology, and in particular to techniques for a machine learning framework for breast cancer histologic grading.

Breast cancer is the most common cancer in women and one of the leading causes of cancer death worldwide. The heterogeneous nature of breast cancer makes its initial characterization a critical step in treatment planning and decision making. One aspect of breast cancer characterization that remains central to its prognostic classification is the Nottingham combined histologic grade. The Nottingham grading system (NGS) is comprised of three components: mitotic count (MC), nuclear pleomorphism (NP), and tubule formation (TF), and is an important component of existing prognostic tools. However, while the combined histologic grade has been repeatedly shown to be associated with clinical outcomes, the task's inherent subjectivity can also result in inter-pathologist variability that limits the generalizability of its prognostic utility. In addition, up to half of breast cancer cases are classified in routine practice as grade 2, an intermediate risk group with limited clinical value due to inclusion of some low and high grade tumors.

The application of computer vision and artificial intelligence (AI) to histopathology has seen tremendous growth in recent years and offers the potential to augment pathologist expertise and increase consistency and efficiency. Work relevant to breast cancer includes AI systems for counting mitoses, scoring nuclear pleomorphism, detecting metastases in lymph nodes, identifying biomarker status, and predicting prognosis. Understanding the performance and application of such tools in the context of existing challenges for pathological review and workflows remains an important next step for translation to clinical utility.

### BRIEF SUMMARY OF THE INVENTION

In various embodiments, a computer-implemented method comprises: accessing a whole slide image of a specimen, wherein the image comprises a depiction of cells corresponding to a disease; processing the image using a first machine learning process, wherein a first output of the first machine learning process corresponds to a mask indicating particular portions of the image predicted to depict the tumor cells; applying the mask to the image to generate a masked image; processing the masked image using a second machine learning process, wherein a second output of the second machine learning process corresponds to a mitotic count predicted score for a mitotic count depicted in the image; processing the masked image using a third machine learning process, wherein a third output of the third machine learning process corresponds to a nuclear pleomorphism predicted score for nuclear pleomorphism depicted in the image; processing the masked image using a fourth machine learning process, wherein a fourth output of the fourth machine learning process corresponds to a tubule formation predicted score for tubule formation depicted in the image; generating a combined score of a predicted histologic grade of the disease in the image based on the second output, the third output, and the fourth output; and outputting the combined score of the predicted histologic grade.

In some embodiments, the first machine learning process comprises a first machine learning model that segments tumor cells in the image to generate the mask.

In some embodiments, the second machine learning process comprises: generating a first set of patches of the image, wherein each patch of the first set of patches corresponds to a portion of the image; generating, for each patch of the first set of patches, a mitotic count patch-level score by inputting the patch into a second machine learning model, wherein the mitotic count patch-level score corresponds to a likelihood of the patch corresponding to a mitotic figure; determining a plurality of metrics corresponding to mitotic density of the image based on the mitotic count patch-level score for each patch of the first set of patches; and generating the mitotic count predicted score for the image by inputting the plurality of metrics into a third machine learning model.

In some embodiments, the third machine learning process comprises: generating a second set of patches of the image, wherein each patch of the second set of patches corresponds to a portion of the image; generating, for each patch of the second set of patches, a nuclear pleomorphism patch-level score by inputting the patch into a fourth machine learning model, wherein the nuclear pleomorphism patch-level score corresponds to a likelihood of the patch corresponding to each grade score of a plurality of grade scores associated with nuclear pleomorphism; determining a metric associated with each grade score of the plurality of grade scores; and generating the nuclear pleomorphism predicted score for the image by inputting the metric associated with each grade score of the plurality of grade scores into a fifth machine learning model.

In some embodiments, the fourth machine learning process comprises: generating a third set of patches of the image, wherein each patch of the third set of patches corresponds to a portion of the image; generating, for each patch of the third set of patches, a tubule formation patch-level score by inputting the patch into a sixth machine learning model, wherein the tubule formation patch-level score corresponds to a likelihood of the patch corresponding to each grade score of a plurality of grade scores associated with tubule formation; determining a metric associated with each grade score of the plurality of grade scores; and generating the tubule formation predicted score for the image by inputting the metric associated with each grade score of the plurality of grade scores into a seventh machine learning model.

In some embodiments, the first machine learning process, the second machine learning process, the third machine learning process, and the fourth machine learning process comprise a convolutional neural network.

In some embodiments, the combined score comprises a continuous score between a first value and a second value.

In some embodiments, the computer-implemented method further comprises characterizing, classifying, or a combination thereof, the image with respect to the disease based on the combined score; and outputting, an inference based on the characterizing, classifying, or the combination thereof.

In some embodiments, the computer-implemented method further comprises determining a diagnosis of a subject associated with the image, wherein the diagnosis is determined based on the inference.

In some embodiments, the computer-implemented method further comprises administering a treatment to the subject based on (i) the inference and/or (ii) the diagnosis of the subject.

In some embodiments, a system is provided that includes one or more data processors and a non-transitory computer readable storage medium containing instructions which, when executed on the one or more data processors, cause the one or more data processors to perform part or all of one or more methods disclosed herein.

In some embodiments, a computer-program product is provided that is tangibly embodied in a non-transitory machine-readable storage medium and that includes instructions configured to cause one or more data processors to perform part or all of one or more methods disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

Aspects and features of the various embodiments will be more apparent by describing examples with reference to the accompanying drawings, in which:

FIG. 1 shows an exemplary system for generating digital pathology images in accordance with various embodiments.

FIG. 2 shows a manual annotation and grading process for digital pathology images in accordance with various embodiments.

FIG. 3 shows a diagram that illustrates processing digital pathology images using a deep learning system in accordance with various embodiments.

FIG. 4 illustrates a block diagram of an example of determining an overall histologic score for an image in accordance with various embodiments.

FIG. 5 shows a block diagram that illustrates a computing environment for processing digital pathology images using a deep learning system in accordance with various embodiments.

FIG. 6 shows a flowchart illustrating a process for using a deep learning system for histologic grading in accordance with various embodiments.

FIG. 7 depicts test sets used for prognostic analysis and evaluation of performance for grading algorithms in accordance with various embodiments.

FIG. 8 illustrates example classifications from individual models of a deep learning system in accordance with various embodiments.

FIG. 9 illustrates examples of patch-level predictions across entire whole slide images for nuclear pleomorphism and tubule formation in accordance with various embodiments.

FIG. 10 illustrates an assessment of slide-level classification of nuclear pleomorphism and tubule formation by pathologists and the deep learning system in accordance with various embodiments.

FIG. 11 illustrates inter-pathologist and deep learning system-pathologist concordance for slide-level component scoring in accordance with various embodiments.

FIG. 12 illustrates full confusion matrices for inter-pathologist agreement and for deep learning system agreement with the majority vote scores at the region-level in accordance with various embodiments.

FIG. 13 illustrates full confusion matrices for inter-pathologist agreement and for deep learning system agreement with the majority vote scores at the slide-level in accordance with various embodiments.

FIG. 14 illustrates full confusion matrices for inter-pathologist agreement and for deep learning system agreement with the majority vote scores at the patch-level in accordance with various embodiments.

FIG. 15 illustrates correlation between mitotic count scores and Ki-67 gene expression for mitotic count scores provided by a deep learning system and by a pathologist in accordance with various embodiments.

### DETAILED DESCRIPTION OF THE INVENTION

While certain embodiments are described, these embodiments are presented by way of example only, and are not intended to limit the scope of protection. The apparatuses, methods, and systems described herein may be embodied in a variety of other forms. Furthermore, various omissions, substitutions, and changes in the form of the example methods and systems described herein may be made without departing from the scope of protection.

### I. Overview

Histologic grading of digital pathology images provides a metric for assessing a presence and degree of disease. In particular, the Nottingham grading system is conventionally employed for histologic grading of breast cancer. The Nottingham grading system involves reviewing and scoring histologic features of mitotic count, nuclear pleomorphism, and tubule formation. Mitotic count is a measure of how fast cancer cells are dividing and growing. Nuclear pleomorphism is a measure of the extent of abnormalities in the appearance of tumor nuclei. Tubule formation describes the percentage of cells that have tube-shaped structure. In general, a higher mitotic count, nuclear pleomorphism, and/or tubule formation corresponds to a higher histologic grade, which is measured as a score of 1, 2, or 3 for each histologic feature.

Conventionally, histologic grading is performed by manual analysis by pathologists, or other technicians. As such, there is inherent subjectivity resulting in inter-pathologist variability. This can limit the ability to generalize the prognostic utility of the histologic grade. In addition, machine learning models that have been developed for characterizing digital pathology images associated with breast cancer typically focus on one or two of the histologic features, but do not account for each of mitotic count, nuclear pleomorphism, and tubule formation. As a result, the predicted histologic grade for an image may be inaccurate.

To address these issues and others, various embodiments disclosed herein are directed to methods, systems, and computer readable storage media to use a deep learning system with machine learning processes for each of mitotic count, nuclear pleomorphism, and tubule formation to predict a histologic grade for an image. A first stage of each machine learning process can be at a patch level, and an output of the first stage can be used in a second stage at an image level. Predicted scores can be generated for each of the histologic features, which can then be combined to generate a combined score of the predicted histologic grade for the image. Since the predicted histologic grade provided by the deep learning system can be more accurate compared to conventional systems, the deep learning system may additionally facilitate improved diagnosis, prognosis, and treatment decisions that are made based on the predicted histologic grade.

In one illustrative embodiment, a computer-implemented process is provided that comprises: accessing a whole slide image of a specimen, where the image comprises a depiction of cells corresponding to a disease; processing the image using a first machine learning process, where a first output of the first machine learning process corresponds to a mask indicating particular portions of the image predicted to depict the tumor cells; applying the mask to the image to generate a masked image; processing the masked image using a second machine learning process, wherein a second output of the second machine learning process corresponds to a mitotic count predicted score for a mitotic count depicted in the image; processing the masked image using a third machine learning process, wherein a third output of the third machine learning process corresponds to a nuclear pleomorphism predicted score for nuclear pleomorphism depicted in the image; processing the masked image using a fourth machine learning process, wherein a fourth output of the fourth machine learning process corresponds to a tubule formation predicted score for tubule formation depicted in the image; generating a combined score of a predicted histologic grade of the disease in the image based on the second output, the third output, and the fourth output; and outputting the combined score of the predicted histologic grade.

### II. Generation of Digital Pathology Images

Digital pathology involves the interpretation of digitized images in order to correctly diagnose subjects and guide therapeutic decision making. Digital pathology solutions may involve automatically detecting or classifying biological objects of interest (e.g., positive, negative tumor cells, etc.). Tissue slides can be obtained and scanned, and then image analysis can be performed to detect, quantify, and classify the biological objects in the image. Preselected areas or the entirety of the tissue slides may be scanned with a digital image scanner (e.g., a whole slide image (WSI) scanner) to obtain the digital images, and the image analysis may be performed using one or more image analysis algorithms.

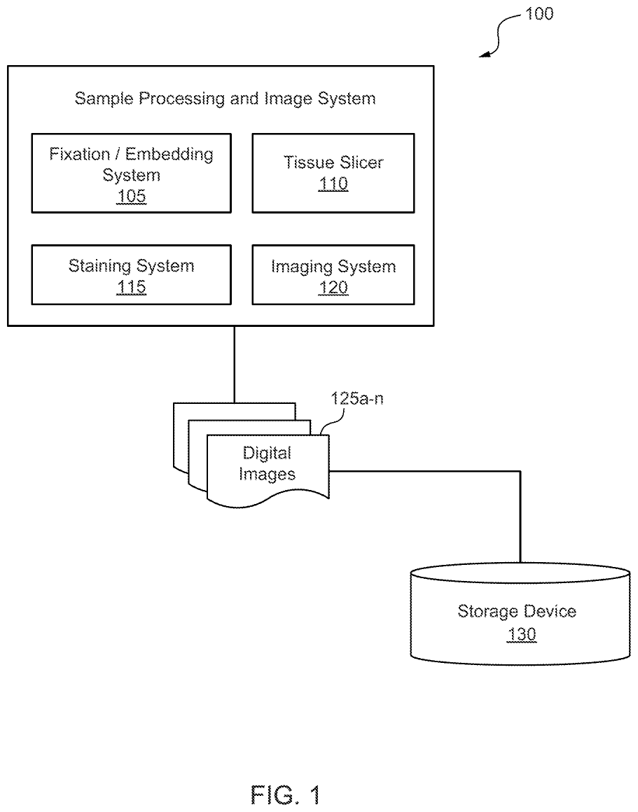

FIG. 1 shows an exemplary system 100 for generating digital pathology images. A fixation/embedding system 105 can fix and/or embed a tissue sample (e.g., a sample including at least part of at least one tumor) using a liquid fixing agent (e.g., a formaldehyde solution) and/or an embedding substance (e.g., a histological wax, such as a paraffin wax and/or one or more resins, such as styrene or polyethylene). The sample can be exposed to the fixating agent for a predefined period of time (e.g., at least 3 hours) and then dehydrated (e.g., via exposure to an ethanol solution and/or a clearing intermediate agent). The embedding substance can permeate the sample when it is in liquid state (e.g., when heated).

A tissue slicer 110 may then be used for sectioning the fixed and/or embedded tissue sample (e.g., a sample of a tumor). Sectioning involves cutting slices (e.g., a thickness of, for example, 4-5 μm) of a sample from a tissue block for the purpose of mounting the slice on a microscope slide for examination. A microtome, vibratome, or compresstome may be used to perform the sectioning. Tissue may first be frozen rapidly in dry ice or Isopentane, and then cut in a refrigerated cabinet (e.g., a cryostat) with a cold knife. Liquid nitrogen may alternatively be used to freeze the tissue. In some cases, sections can be embedded in an epoxy or acrylic resin, which may enable thinner sections (e.g., <2 μm) to be cut. The sections may then be mounted on one or more glass slides with a coverslip placed on top to protect the sample section.

Tissue sections may be stained so that the cells within them, which are virtually transparent, can become more visible. In some instances, the staining is performed manually, or the staining may be performed semi-automatically or automatically using a staining system 115. The staining process includes exposing sections of tissue samples or of fixed liquid samples to one or more different stains (e.g., consecutively or concurrently) to express different characteristics of the tissue.

For example, staining may be used to mark particular types of cells and/or to flag particular types of nucleic acids and/or proteins to aid in the microscopic examination. A dye or stain is added to a sample to qualify or quantify the presence of a specific compound, a structure, a molecule, or a feature (e.g., a subcellular feature). For example, stains can help to identify or highlight specific biomarkers from a tissue section. In other example, stains can be used to identify or highlight biological tissues (e.g., muscle fibers or connective tissue), cell populations (e.g., different blood cells), or organelles within individual cells.

One exemplary type of tissue staining is histochemical staining, which uses one or more chemical dyes (e.g., acidic dyes, basic dyes, chromogens) to stain tissue structures. Histochemical staining may be used to indicate general aspects of tissue morphology and/or cell microanatomy (e.g., to distinguish cell nuclei from cytoplasm, to indicate lipid droplets, etc.). One example of a histochemical stain is H&E. Other examples of histochemical stains include trichrome stains (e.g., Masson's Trichrome), Periodic Acid-Schiff (PAS), silver stains, and iron stains.

Another type of tissue staining is IHC, also called “immunohistochemical staining”, which uses a primary antibody that binds specifically to the target biomarker (or antigen) of interest. IHC may be direct or indirect. In direct IHC, the primary antibody is directly conjugated to a label (e.g., a chromophore or fluorophore). In indirect IHC, the primary antibody is first bound to the target biomarker, and then a secondary antibody that is conjugated with a label (e.g., a chromophore or fluorophore) is bound to the primary antibody.

After staining, the sections may then be mounted on slides, which an imaging system 120 can then scan or image to generate raw digital-pathology images 125a-n. A microscope (e.g., an electron, optical, or confocal microscope) can be used to magnify the stained sample. For example, optical microscopes, electron microscopes, or confocal microscopes may be used. An imaging device (combined with the microscope or separate from the microscope) images the magnified biological sample to obtain the image data. The image data may be a multi-channel image (e.g., a multi-channel fluorescent) with several channels, a z-stacked image (e.g., the combination of multiple images taken at different focal distances), or a combination of multi-channel and z-stacking. The imaging device may include, without limitation, a camera (e.g., an analog camera, a digital camera, etc.), optics (e.g., one or more lenses, sensor focus lens groups, microscope objectives, etc.), imaging sensors (e.g., a charge-coupled device (CCD), a complimentary metal-oxide semiconductor (CMOS) image sensor, or the like), photographic film, or the like. An image sensor, for example, a CCD sensor can capture a digital image of the biological sample. In some embodiments, the imaging device is a brightfield imaging system, a multispectral imaging (MSI) system or a fluorescent microscopy system. The imaging device may utilize nonvisible electromagnetic radiation (UV light, for example) or other imaging techniques to capture the image. The image data received by the analysis system may be raw image data or derived from the raw image data captured by the imaging device.

The digital images 125a-n of the stained sections may then be stored in a storage device 130 such as a server. The images may be stored locally, remotely, and/or in a cloud server. Each image may be stored in association with an identifier of a subject and a date (e.g., a date when a sample was collected and/or a date when the image was captured). During analysis, an image may further be transmitted to another system (e.g., a system associated with a pathologist, an automated or semi-automated image analysis system, or a machine learning training and deployment system, as described in further detail herein).

It will be appreciated that modifications to processes described with respect to system 100 are contemplated. For example, if a sample is a liquid sample, embedding and/or sectioning may be omitted from the process.

### III. Exemplary Slide-Level and Region-Level Annotations

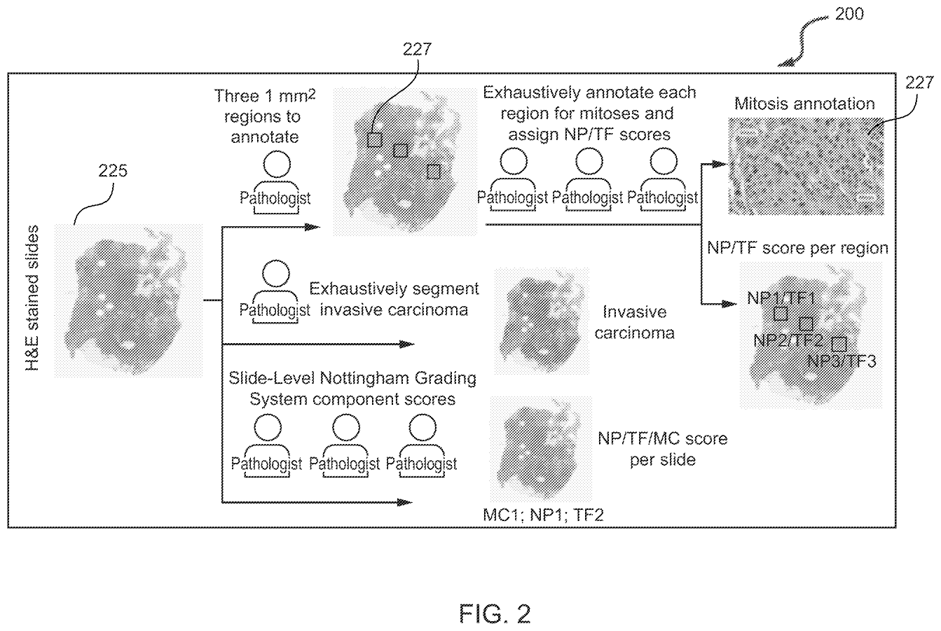

FIG. 2 shows a manual annotation and grading process 200 for digital pathology images. In some cases, one or more pathologists can provide the manual annotation and grading for a digital pathology image, where the image can be at the slide-level 225 or the region-level 227. The slide-level 225 refers to an image where the whole section mounted on the slide is visible for annotation. Whereas the region-level 227 refers to images of a smaller portion/region of the whole section, where sometimes the image is a higher magnification of the portion/region. For example, at the slide-level 225, a pathologist can annotate regions of interest by applying a bounding box around the regions. A single whole section may have multiple regions of interest bounded, as depicted in FIG. 2. The bounding box is illustrated as being 1 mm², but the bounding box may be a different size in other examples. Further, the pathologists can provide annotations at a slide-level and region-level for all components of the histologic grade (e.g., mitotic count, nuclear pleomorphism, and tubule formation). In addition, pathologists may segment invasive carcinomas in the image and provide slide-level 225 histologic grading scores (e.g., between 1 and 3) for each component of the histologic grade. In an example in which multiple pathologists provide histologic grading scores, a majority voting technique may be employed to determine the histologic grading scores if the pathologists disagree. That is, if two pathologists indicate a mitotic count histologic grading score of 1 for the image, but a third pathologist indicates a mitotic count histologic grading score of 2, the majority voting technique can result in the mitotic count histologic grading score of the image being determined as 1.

At the region-level, each region 227 identified by the pathologist can be further annotated with respect to each of the components of the histologic grade. This allows multiple pathologists to exhaustively annotate (e.g., at the cell-level) each region 227 for mitosis and assign histologic grading scores for nuclear pleomorphism and tubule formation for each region 227. Cells in the region 227 that appear to be actively dividing are assigned a “mitosis” label.

### IV. Exemplary Techniques and Systems for Histologic Grading

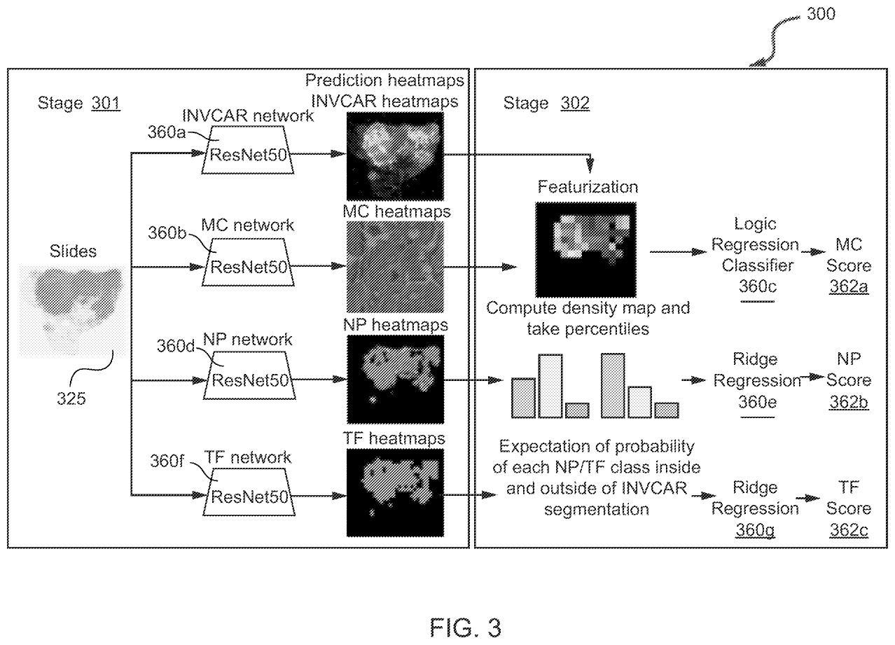

FIG. 3 shows a diagram that illustrates processing digital pathology images using a deep learning system 300 in accordance with various embodiments. As illustrated, a slide/image 325 is processed using multiple machine learning models 360a-g to generate an overall histologic grading score for the slide. The machine learning models are split into a first stage 301 and a second stage 302 where first stage models are paired with second stage models forming a machine learning process. For example, the first stage MC network model 360b is paired with the second stage logic regression classifier model 360c to form a machine learning process that predicts a score for mitotic count. Accordingly, each histological component has its own corresponding machine learning process, comprising a first stage model and a second stage model. The first stage model performs histologic grading at the patch-level to generate a patch-level score that is then input into its paired second stage model. Meanwhile, the paired second stage model will perform histologic grading at the slide-level and generate the corresponding predicted histologic score.

As illustrated in FIG. 3, the deep learning system 300 is comprised of a first, second, third, and fourth machine learning processes, where the second, third and fourth processes correspond to a component of histologic grading (e.g., mitotic count, nuclear pleomorphism, and tubule formation respectively). Initially, the first machine learning process uses the first machine learning model (i.e., the INVCAR network 360a) to segment invasive carcinoma regions on slide/image 325 and generate tumor masks (also referred to as “masks”) indicating portions of the image/slide 325 predicted to depict tumor cells. The tumor masks are output as heatmaps where the colors correspond to a predicted likelihood of a region of the slide/image 325 depicting an invasive carcinoma. Then the tumor masks are applied to slide/image 325 and those regions that contain cancer cells are output to the first stage machine learning models (360b, d, and f) to process slide/image 325 into sets of patches. Alternatively, the tumor masks are applied to slide/image 325 and the first stage machine learning models process the whole slide image into sets of patches and once the patch-level scores are generated, those patches not predicted to be associated with tumor cells are removed.

Once generated, sets of patches are input into the first stage MC network model 360b (i.e., the second machine learning model associated with the mitotic count component of histologic grading) which outputs heatmaps for each patch with colors corresponding to the predicted likelihoods that the regions depict a mitotic figure. The heatmaps are used to determine a patch-level score for the set of patches, and the patch-level score is input into the second stage logic regression classifier model 360c (i.e., the third machine learning model) to determine a predicted score (e.g., between 1 and 3) for mitotic count 362a in the image. In addition, the sets of patches generated from masked slide/image 325 can also be input into the NP network model 360d (i.e., the fourth machine learning model associated with the nuclear pleomorphism component of histologic grading) and the TF network model 360f (i.e., the sixth machine learning model associated with the tubule formation component of histologic grading). Both learning models, NP network model 360d and TF network model 360f, will also generate a heatmap with colors corresponding to predicted likelihoods that the regions depict either nuclear pleomorphisms or tubule formation, respectively. The patch-level score associated with nuclear pleomorphisms will be input into the ridge regression model 360e (i.e., the fifth machine learning model) and the patch-level score associated with tubule formation will be input into the ridge regression model 360g (i.e., the seventh machine learning model) to determine a predicted score (e.g., between 1 and 3) for nuclear pleomorphisms 362b or tubule formation 362c, respectively, for the image. The three predicted scores 362a-c may then be combined to determine an overall histologic score for the slide 325 with respect to a disease (e.g., breast cancer).

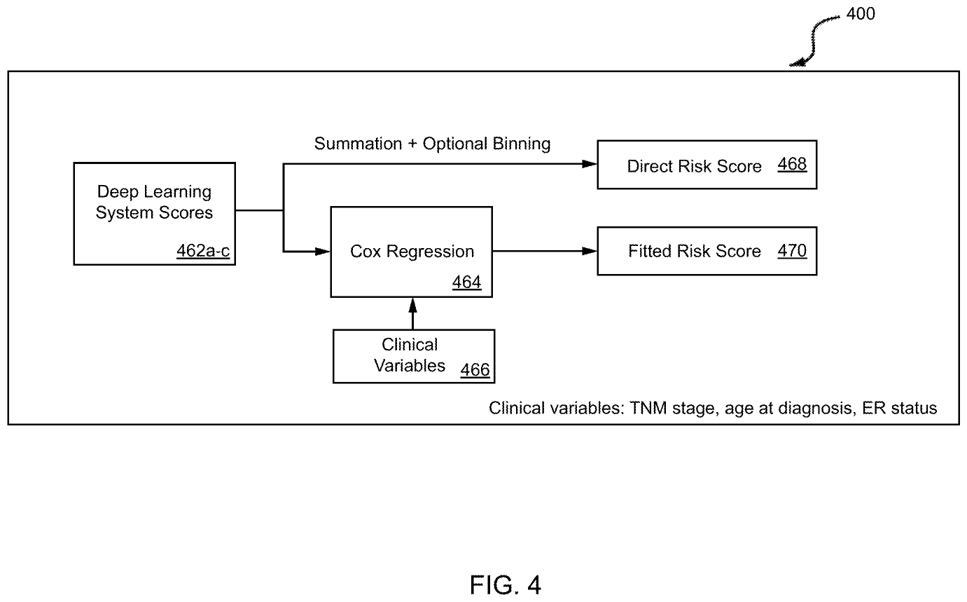

FIG. 4 illustrates a block diagram 400 of an example for determining an overall histologic score for an image, based on the predicted scores from the deep learning system described in FIG. 3, in accordance with various embodiments. The overall histologic score accounts for mitotic count, nuclear pleomorphism, and tubule formation depicted in the slide/images described in FIG. 3. In an example, once the deep learning system predicted scores 462a-c for each of mitotic count, nuclear pleomorphism, and tubule formation are determined by various machine learning models, a direct risk score 468 or a fitted risk score 470 can be generated. The direct risk score 468 and the fitted risk score 470 can be combined scores representing a histologic grade of the image.

In an example, generating the direct risk score 468 can involve summation and an optional binning of the predicted scores 462a-c. So, if the predicted score 462a for mitotic count is 2, the predicted score 462b for tubule formation is 1, and the predicted score 462c for nuclear pleomorphism is 2, the direct risk score 468 can be 5. Optionally, the resulting summation can be binned into one of three bins, where a bin corresponds to a Nottingham histological grade (i.e., grade I, grade II, or grade III). The first bin is for tumors that received a grade I, indicating the summation of their predicted scores 462a-c is between 3-5. The second bin for grade II tumors has a summation between 6-7, and the third bin for grade III tumors has a summation between 8-9. In some instances, each of the predicted scores 462a-c generated by the machine learning models can be a continuous score between 1 and 3 (e.g., 1.5, 2.3, etc.) rather than an integer value. So, the direct risk score 468 can be a continuous value between 3 and 9.

In some examples, the predicted scores 462a-c, along with clinical variables 466 (e.g., age, tumor, node, metastasis (TNM) status, estrogen receptor status (e.g., positive or negative), etc.) can be input into a Cox regression (or proportional hazards regression) model 464 that generates the fitted risk score 470 based on the predicted scores 462a-c and the clinical variables 466. As such, the fitted risk score 470 may combine strengths of machine learning with existing knowledge about the prognostic value of morphological features.

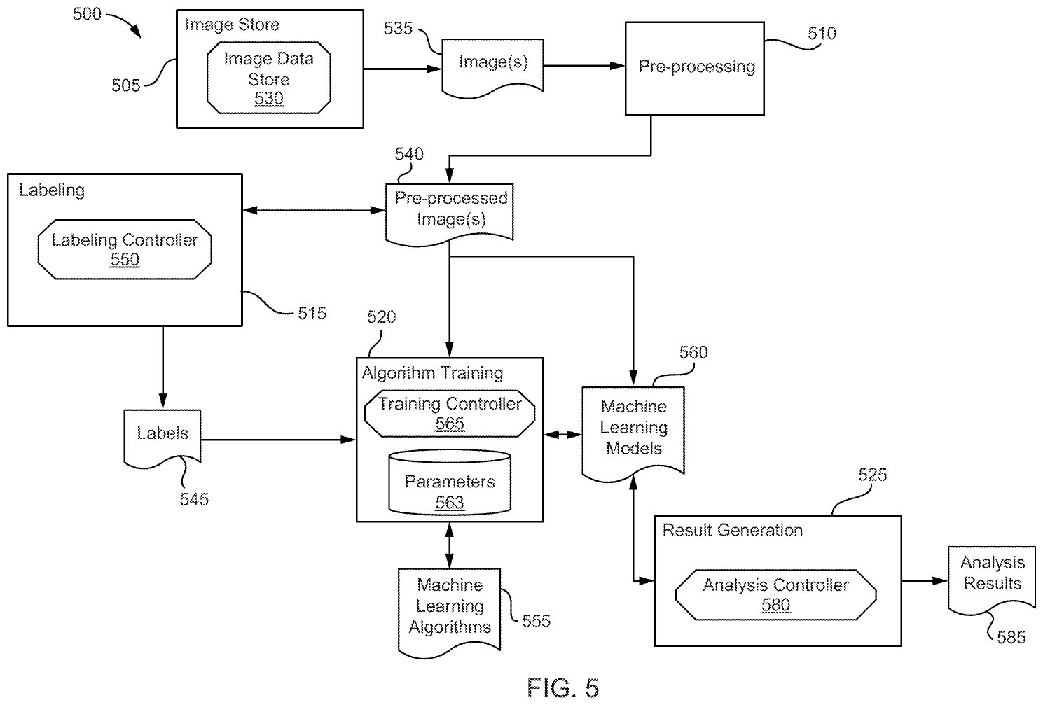

FIG. 5 shows a block diagram that illustrates a computing environment 500 for processing digital pathology images using a deep learning system (e.g., one or more machine learning models) in accordance with various embodiments. As further described herein, processing digital pathology images can include using digital pathology images to train one or more machine learning algorithms and/or transforming part or all of the digital pathology images into one or more results using a trained (or partly trained) version of the machine learning algorithms (i.e., machine learning models).

As shown in FIG. 5, computing environment 500 includes several stages: an image store stage 505, a pre-processing stage 510, a labeling stage 515, a training stage 520, and a result generation stage 525.

The image store stage 505 includes one or more image data stores 530 (e.g., storage device 130 described with respect to FIG. 1) that stores a set of digital images 535 comprising slide-level (e.g., showing the entire sample on the slide) or region-level (e.g., regions of interest as described with respect to FIG. 2) images of a biological sample (e.g., tissue slides) that are accessed by the pre-processing stage 510. Each digital image 535 stored in each image data store 530 and accessed at image store stage 505 may include a digital pathology image generated in accordance with part or all of processes described with respect to system 100 depicted in FIG. 1. In some embodiments, each digital image 535 includes image data from one or more scanned slides. Each of the digital images 535 may correspond to image data from a single specimen and/or a single day on which the underlying image data corresponding to the image was collected.

The image data may include an image 535 and information related to color channels or color wavelength channels, as well as details regarding the imaging platform on which the image was generated. For instance, a tissue section may be stained using a staining assay containing one or more different biomarkers associated with a disease (e.g., breast cancer). Example biomarkers can include biomarkers for estrogen receptors (ER), human epidermal growth factor receptors 2 (HER2), human Ki-67 protein, progesterone receptors (PR), programmed cell death protein 1 (PD1), and the like, where the tissue section is detectably labeled with binders (e.g., antibodies) for each of ER, HER2, Ki-67, PR, PD1, etc. A tissue section may be processed in an automated staining/assay platform that applies a staining assay to the tissue section, resulting in a stained sample. In some examples, the tissue section may be stained with hematoxylin and eosin. Stained tissue sections may be supplied to an imaging system, for example to a microscope or a whole-slide scanner having a microscope and/or imaging components.

At the pre-processing stage 510, the one or more sets of digital images 535 are pre-processed using one or more techniques to generate a corresponding pre-processed image 540. The pre-processing may comprise cropping the images. In some instances, the pre-processing may further involve normalization to put all features on a same scale (e.g., size scale, color scale, or a color saturation scale). In some instances, the images may be resized while keeping with the original aspect ratio. The pre-processing may further involve removing noise, such as by applying a Gaussian function or Gaussian blur.

The pre-processed images 540 may include one or more training images, validation images, and unlabeled images. The pre-processed images 540 can be accessed at various times and by the various stages of computing environment 500. For example, an initial set of training and validation pre-processed images 540 may first be accessed at the labeling stage 515 to assign labels to the pre-processed images 540 before being input into the algorithm training stage to be used for training machine learning algorithms 555. Another example includes the training and validation pre-processed images 540 being accessed directly at the algorithm training stage 520 and used to train machine learning algorithms 555 with unlabeled pre-processed images. Further, unlabeled input images may be subsequently accessed (e.g., at a single or multiple subsequent times) and used by trained machine learning models 560 to provide desired output (e.g., cell classification).

In some instances, the machine learning algorithms 555 are trained using supervised training where some or all of the pre-processed images 540 are partly or fully labeled manually, semi-automatically, or automatically at labeling stage 515. The labels 545 identify a “correct” interpretation (i.e., the “ground-truth”) of various biomarkers and cellular/tissue structures within the pre-processed images 540. For example, the label 545 may identify a feature of interest (for example) a mitotic count score, a nuclear pleomorphism score, a tubule formation score, a categorical characterization of a slide-level or region-specific depiction (e.g., that identifies a specific type of cell), a number (e.g., that identifies a quantity of a particular type of cells within a region, a quantity of depicted artifacts, or a quantity of necrosis regions), presence or absence of one or more biomarkers, etc. In some instances, a label 545 includes a location. For example, a label 545 may identify a point location of a nucleus of a cell of a particular type or a point location of a cell of a particular type (e.g., raw dot labels). As another example, a label 545 may include a border or boundary, such as a border of a depicted tumor, blood vessel, necrotic region, etc. Depending on a feature of interest, a given labeled pre-processed image 540 may be associated with a single label 545 or multiple labels 545. In the latter case, each label 545 may be associated with, for example, an indication as to which position or portion within the pre-processed image 540 the label corresponds.

A label 545 assigned at labeling stage 515 may be identified based on input from a human user (e.g., pathologist or image scientist) and/or an algorithm (e.g., an annotation tool) configured to define a label 545. In some instances, labeling stage 515 can include transmitting and/or presenting part or all of one or more pre-processed images 540 to a computing device operated by the user. In some instances, labeling stage 515 includes availing an interface (e.g., using an API) to be presented by labeling controller 550 on the computing device operated by the user, where the interface includes an input component to accept input that identifies labels 545 for features of interest. For example, a user interface may be provided by the labeling controller 550 that enables selection of an image or region of an image for labeling. One or more users operating the terminal may select an image using the user interface and provide annotations for each histologic feature of the Nottingham grading system. That is, the users can provide annotations for mitotic count, tubule formation, and nuclear pleomorphism for each image. Several image selection mechanisms may be provided, such as designating known or irregular shapes, or defining an anatomic region of interest (e.g., tumor region). The users operating the terminal may select one or more labels 545 to be applied to the selected image such as a point location of a cell, a positive indicator for a biomarker expressed by a cell, a negative indicator for a biomarker not expressed by a cell, a boundary around a cell, and the like. In some instances, labeling stage 515 includes labeling controller 550 implementing an annotation algorithm in order to semi-automatically or automatically label various features of an image or a region of interest within the image.

Moreover, a user may identify regions of interest (e.g., 1 mm by 1 mm regions) within an image and annotate each identified region with labels 545. For example, the users may identify cells undergoing mitosis in the regions of interest and can add labels 545 to each cell identified as mitotic. By counting the number of mitotic cells, a mitotic count score (e.g., 1-3) is determined for that region. Rather than instances of tubule formation and nuclear pleomorphism also being exhaustively annotated, one or more users can provide the labels 545 for each identified regions for these histologic features. In addition, the one or more users can also provide labels 545 at the image-level for each histologic feature. Accordingly, each image and each identified region is associated with labels 545 of a mitotic count score, a nuclear pleomorphism score, and a tubule formation score.

At training stage 520, labels 545 and corresponding pre-processed images 540 can

be used by the training controller 565 to train machine learning algorithm(s) 555 in accordance with the various workflows described herein. For example, to train an algorithm 555, the pre-processed images 540 may be split into a subset of images 540a for training (e.g., 90%) and a subset of images 540b for validation (e.g., 10%). The splitting may be performed randomly (e.g., a 90/10% or 70/30%) or the splitting may be performed in accordance with a more complex validation technique such as K-Fold Cross-Validation, Leave-one-out Cross-Validation, Leave-one-group-out Cross-Validation, Nested Cross-Validation, or the like to minimize sampling bias and overfitting.

In some instances, the machine learning algorithms 555 make up a deep learning system that includes convolution neural networks (CNNs), modified CNNs with encoding layers substituted by a residual neural network (“Resnet”), or modified CNNs with encoding and decoding layers substituted by a Resnet. In other instances, the machine learning algorithms 555 can be any suitable machine learning algorithms configured to localize, classify, and or analyze pre-processed images 540, such as a two-dimensional CNNs (“2DCNN”). Mask R-CNNs, U-Nets, etc., or combinations of one or more of such techniques. The computing environment 500 may employ the same type of machine learning processes of machine learning algorithms or different types of machine learning processes trained to detect and classify different histologic components. For example, computing environment 500 can include a first machine learning process (e.g., a CNN) for segmenting the invasive carcinomas. Further, the computing environment 500 can also include a second machine learning process (e.g., a CNN and linear regression classifier) for detecting and classifying mitotic count. The computing environment 500 can also include a third machine learning process (e.g., a CNN and ridge regression) for detecting and classifying nuclear pleomorphism. Similarly, the computing environment 500 can also include a fourth machine learning process (e.g., a CNN and ridge regression) for detecting and classifying tubule formation.

The training process for the machine learning algorithms 555 includes selecting hyperparameters for the machine learning algorithms 555 from a parameter data store 563, inputting the subset of images 540a (e.g., labels 545 and corresponding pre-processed images 540) into the machine learning algorithms 555, and performing iterative operations to learn a set of parameters (e.g., one or more coefficients and/or weights) for the machine learning algorithms 555. The hyperparameters are settings that can be tuned or optimized to control the behavior of the machine learning algorithm 555.

In some instances, the trained machine learning models 560 may be used to generate masks that identify a location of depicted cells associated with one or more biomarkers. For example, given a tissue stained for a single biomarker, the trained machine learning models 560 may include a segmentation machine learning model (e.g., a CNN) configured to segment tumor cells in an image and generate a mask indicating particular portions of the image predicted to depict tumor cells. The mask can be applied to the image before the image is processed with the other trained machine learning models 560. The trained machine learning models 560 may further be configured to detect, characterize, classify, or a combination thereof, the image with respect to a disease.

Patches can be generated for each of the images 540a, and the patches can be input into the first stage machine learning models of the second, third, and fourth machine learning processes along with the labels 545 (the machine learning models 360b, d, and fin stage 301 described in FIG. 3). A patch refers to a container of pixels corresponding to a portion of a whole image, a whole slide, or a whole mask. The patch has (x, y) pixel dimensions (e.g., 256 pixels by 256 pixels). Based on the patches and the labels 545, the first stage machine learning models generate patch-level scores for mitotic count, nuclear pleomorphism, and tubule formation. For instance, masked image patches and labels 545 associated with mitotic count can be input into the first stage machine learning model of the second machine learning process (i.e., MC network model 360b shown in FIG. 3), masked image patches and labels 545 associated with nuclear pleomorphism can be input into the first stage machine learning model of the third machine learning process (i.e., NP network model 360d shown in FIG. 3), and the masked image patches and labels 545 associated with tubule formation can be input into the first stage machine learning model of the fourth machine learning process (i.e., TF network model 360f shown in FIG. 3). The output of the first stage machine learning model of the second machine learning process can be a mitotic count patch-level score that corresponds to a likelihood of the patch corresponding to a mitotic figure. The outputs of each of the first stage machine learning models of the third machine learning process and the fourth machine learning process can be a nuclear pleomorphism patch-level score and a tubule formation patch level score, respectively. The nuclear pleomorphism patch-level score corresponds to a likelihood of the patch corresponding to each grade score (e.g., 1, 2, and 3) associated with nuclear pleomorphism. The tubule formation patch-level score corresponds to a likelihood of the patch corresponding to each grade score (e.g., 1, 2, and 3) associated with tubule formation.

In some examples, the outputs of the first stage machine learning models are used as inputs to the second stage machine learning models of the machine learning processes (the machine learning models 360c, e, and g in stage 302 described in FIG. 3). While the first stage machine learning models are performed at the patch-level, the second stage machine learning models are performed at the slide-level. For mitotic count, metrics corresponding to mitotic density of the image can be generated based on the mitotic count patch-level score for each patch. The metrics may be the mitotic density values corresponding to particular percentiles for the image. For instance, the percentiles may be the 5^{th}, 25^{th}, 50^{th}, 75^{th}, and 95^{th}-percentiles for the image. The metrics can be input into the second stage machine learning model (i.e., logistic regression classifier model 360c shown in FIG. 3) of the second machine learning process to generate a predicted mitotic count score for the image. For nuclear pleomorphism, a metric associated with each grade score is determined. The metric may be the mean patch-level output (e.g., mean softmax value) for each possible score (e.g., 1, 2, or 3) for the image. The metric associated with each grade score can be input to the second stage machine learning model (i.e., ridge regression model 360e shown in FIG. 3) of the third machine learning process to generate a predicted score for nuclear pleomorphism for the image. Similarly, for tubule formation, a metric associated with each grade score is determined. The metric may be the mean patch-level output (e.g., mean softmax value) for each possible score (e.g., 1, 2, or 3) for the image. The metric associated with each grade score can be input to the second stage machine learning model (i.e., ridge regression model 360g shown in FIG. 3) of the fourth machine learning process to generate a predicted score for tubule formation for the image. The predicted scores for each of mitotic count, nuclear pleomorphism, and tubule formation can be a continuous score (e.g., between 1 and 3) or a discrete score (e.g., 1, 2, or 3).

In some instances, the training process includes iterative operations to find a set of parameters for the machine learning algorithms 555 that minimize a loss function for the machine learning algorithms 555. Each iteration can involve finding a set of parameters for the machine learning algorithms 555 so that the value of the loss function using the set of parameters is smaller than the value of the loss function using another set of parameters in a previous iteration. The loss function can be constructed to measure the difference between the outputs predicted using the machine learning algorithms 555 and the labels 545. Once the set of parameters are identified, the machine learning algorithms 555 have been trained and can be utilized for prediction as designed.

The trained machine learning models 560 can then be used (at result generation stage 525) to process new pre-processed images 540 to generate predictions or inferences such as predict scores for histologic components, predict a diagnosis of disease or a prognosis for a subject such as a patient, or a combination thereof. Once predicted scores for each of mitotic count, nuclear pleomorphism, and tubule formation are generated, the trained machine learning models 560 can generate a combined score of the predicted histologic grade of the disease in the image. For example, the combined score may be a summation of the predicted scores for mitotic count, nuclear pleomorphism, and tubule formation. Or, the trained machine learning models 560 may include a Cox regression model, or another suitable model, that generates the combined score based off of the three predicted scores as well as clinical variables (e.g., age, estrogen receptor status, etc.) for the subject. See FIG. 4 for a more detailed description.

In some instances, an analysis controller 580 generates analysis results 585 that are availed to an entity that requested processing of an underlying image. The analysis result(s) 585 may include information calculated or determined from the output of the trained machine learning models 560 such as the combined score of the predicted histologic grade. Automated algorithms may be used to analyze selected regions of images (e.g., masked images) and generate scores. The analysis controller 580 can generate and output an inference based on the detecting, classifying, and/or characterizing. The inference may be used to determine a diagnosis of the subject. In some embodiments, the analysis controller 580 may further communicate with a computing device associated with a pathologist, physician, investigator (e.g., associated with a clinical trial), subject, medical professional, etc. In some instances, a communication from the computing device includes an identifier for a subject, in correspondence with a request to perform an iteration of analysis for the subject. The computing device can further perform analysis based on the output(s) of the machine learning model and/or the analysis controller 580 and/or provide a recommended diagnosis/treatment for the subject(s).

### V. Techniques for Using Machine Learning Models for Histologic Grading

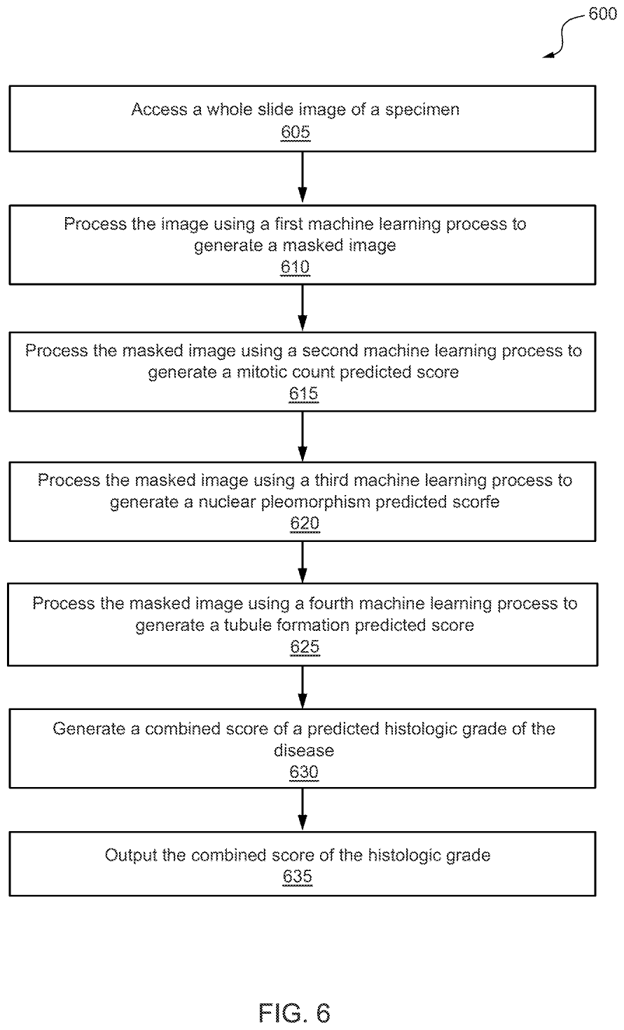

FIG. 6 shows a flowchart illustrating a process 600 for using a deep learning system for histologic grading in accordance with various embodiments. The process 600 depicted in FIG. 6 may be implemented in software (e.g., code, instructions, program) executed by one or more processing units (e.g., processors, cores) of the respective systems, hardware, or combinations thereof. The software may be stored on a non-transitory storage medium (e.g., on a memory device). The process 600 presented in FIG. 6 and described below is intended to be illustrative and non-limiting. Although FIG. 6 depicts the various processing steps occurring in a particular sequence or order, this is not intended to be limiting. In certain alternative embodiments, the steps may be performed in some different order, or some steps may also be performed in parallel.

Process 600 starts at block 605, at which a whole slide image of a specimen is accessed. The image can be generated by a sample processing and image system, as described in FIG. 1. The image may include tumor cells associated with a disease, such as breast cancer. The image can include stains associated with biomarkers of the disease. The image may have a tumor masked applied and be divided into image patches of a predetermined size. For example, the image may be split into image patches having a predetermined size of 64 pixels×64 pixels, 128 pixels×128 pixels, 256 pixels×256 pixels, or 512 pixels×512 pixels.

At block 610, the process 600 involves processing the image using a first machine learning process that comprises a first machine learning model to identify one or more invasive carcinoma regions of the specimen. The output of the first machine learning process can be a mask indicating particular portions of the image predicted to depict the tumor cells. The mask can be applied to the image prior to be processed by the second, third, or fourth machine learning processes to generate predicted scores for histological grade.

At block 615, the process 600 involves processing the masked image using a second machine learning process to generate a mitotic count predicted score. The second machine learning process can involve generating, for each patch, a mitotic count patch-level score by inputting the patch into a second machine learning model (e.g., a CNN). The mitotic count patch-level score can correspond to a likelihood of the patch corresponding to a mitotic figure. Metrics corresponding to mitotic density (e.g., mitotic density values at various percentiles) of the image can be determined based on the mitotic count patch-level score for each patch, and the mitotic count predicted score for the image can be generated by inputting the metrics into a third machine learning model (e.g., linear regression model).

At block 620, the process 600 involves processing the masked image using a third machine learning process to generate a nuclear pleomorphism predicted score. The third machine learning process can involve generating, for each patch, a nuclear pleomorphism patch-level score by inputting the patch into a fourth machine learning model (e.g., a CNN). The nuclear pleomorphism patch-level score corresponds to a likelihood of the patch corresponding to each grade score associated with nuclear pleomorphism. A metric associated with each grade score is determined, and the nuclear pleomorphism predicted score for the image is generated by inputting the metric associated with each grade score into a fifth machine learning model (e.g., ridge regression model).

At block 625, the process 600 involves processing the image using a fourth machine learning process to generate a tubule formation predicted score. The fourth machine learning process can involve generating, for each patch, a tubule formation patch-level score by inputting the patch into a sixth machine learning model (e.g., a CNN). The tubule formation patch-level score corresponds to a likelihood of the patch corresponding to each grade score associated with tubule formation. A metric associated with each grade score is determined, and the tubule formation predicted score for the image is generated by inputting the metric associated with each grade score into a seventh machine learning model (e.g., ridge regression model).

At block 630, the process 600 involves generating a combined score of a predicted histologic grade of the disease. The combined score may be a continuous score or discrete score. The combined score may be a summation or weighted summation of the mitotic count predicted score. the nuclear pleomorphism predicted score, and the tubule formation predicted score. Alternatively, a Cox regression model may receive the mitotic count predicted score, the nuclear pleomorphism predicted score, and the tubule formation predicted score, along with clinical variables to generate the combined score, which can reflect a predicted severity of the disease depicted in the image.

At block 635, the process 600 involves outputting the combined score of the histologic grade. Based on the combined score, the image may be characterized or classified, and an inference based on the characterizing, classifying, or a combination thereof can be output. A diagnosis of a subject associated with the image can be determined based on the inference. A treatment can be determined and administered to the subject associated with the image. In some instances, the treatment can be determined or administered based on the inference output by the machine learning model and/or the diagnosis of the subject.

The combined score of histological grades can further be interpreted by a pathologist, physician, medical professional, or any other qualified personnel to diagnose a patient with a disease (e.g., breast cancer). Qualified personnel take the sum of all three predicted scores output from the second, third, and fourth machine learning processes and assign an overall Nottingham combined score or grade to the tumor. Typically, a higher mitotic count, nuclear pleomorphism, and/or tubule formation corresponds to a higher histologic grade (e.g., such as grade 3), which indicates a high degree of departure from normal breast epithelium. Qualified personnel assign Grade 1 to tumors with a combined score of 5 or less, Grade 2 to tumors with a combined score of 6-7, and Grade 3 to tumors with a combined score of 8-9. Based on the grade assigned to the tumor image, qualified personnel can recommend a treatment option and administer the treatment accordingly.

Additionally or alternatively, the three predicted scores output from the second, third, and fourth machine learning processes can be input into an eighth machine learning algorithm (e.g., a Hidden Markov Model (HMM)) for diagnosis of disease for treatment or a prognosis for a subject such as a patient. Still other types of machine learning algorithms may be implemented in other examples according to this disclosure. The eighth machine learning algorithm is trained to sum the three predicted scores and assign a grade to the tumor based on the scores described above. Further, the eighth machine learning algorithm may have access to clinical variables (e.g., age, tumor, node, metastasis (TNM) status, estrogen receptor status (e.g., positive or negative), etc.) and various treatment plans and survival rates previously administered to a patient to predict a treatment plan, based on the combined tumor grade score, that is most optimal for the patient. Finally, the eighth machine learning algorithm can output the results (combined predict score, tumor grade, treatment options, etc.) to a pathologist, physician, medical professional, patient, etc.

### VI. Examples

The systems and methods implemented in various embodiments may be better understood by referring to the following examples.

### Data

A retrospective study utilized de-identified data from three sources: a tertiary teaching hospital (TTH), a medical laboratory (MLAB), and The Cancer Genome Atlas (TCGA). Whole slide images (WSIs) from TTH included original, archived hematoxylin and eosin (H&E)-stained slides and freshly cut and stained sections from archival blocks. WSIs from MLAB represented freshly cut and H&E stained sections from archival blocks. All WSIs used in the study were scanned at 0.25 μm/pixel (40×). The small number of TCGA images in the breast invasive carcinoma (BRCA) study scanned at 0.50 μm/pixel (20×) were excluded to ensure availability of 40x for deep learning system-based mitotic count and nuclear pleomorphism grading.

All primary invasive breast carcinoma cases with available slides or blocks were reviewed for inclusion. For TTH this included all available cases from 2005-2016, for MLAB this included cases from 2002-2011, and for TCGA data this included the TCGA-BRCA study with cases from 1988-2013.

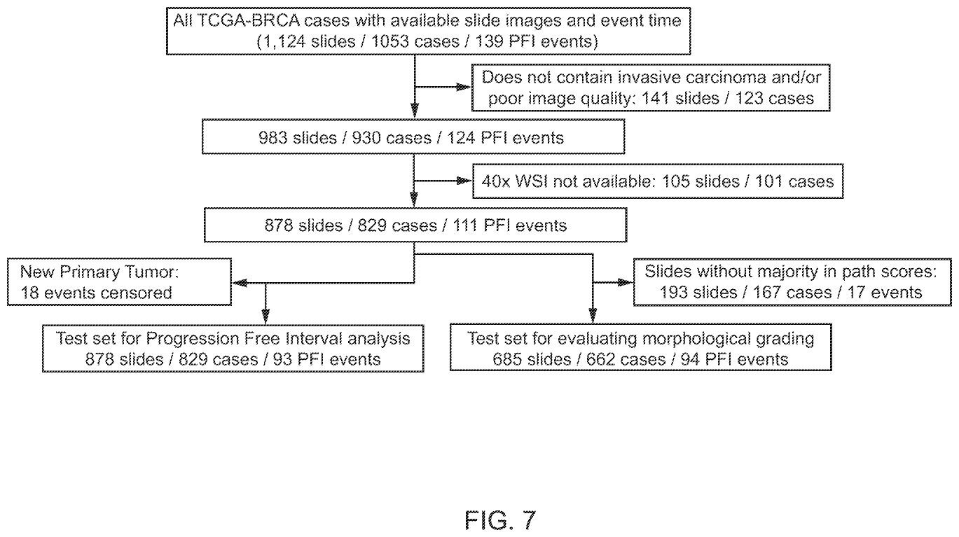

All available WSIs were reviewed by pathologists for slide-level inclusion criteria and quality assurance. Only slides containing H&E-stained primary invasive breast carcinoma from formalin-fixed paraffin-embedded (FFPE) resection specimens were included in the study. Examples of excluded images include lymph node specimens, needle core biopsies, frozen tissue, immunohistochemistry slides, and slides containing carcinoma in-situ only. This resulted in 1,502 slides (657 cases) from TTH, 98 slides (98 cases) from MLAB, and 878 slides (829 cases) from TCGA. The slides from TTH were used for training and tuning of the models, slides from MLAB were used for tuning only, and TCGA slides represent a held-out external test set used only for evaluation of all models. For evaluating the individual machine learning process for each component feature, slides without a pathologist majority were excluded to ensure reliability of the reference for this performance evaluation, resulting in 685 slides/662 cases. Case inclusion and exclusion is summarized in FIG. 7, which depicts TCGA-BRCA test sets used for prognostic analysis (Progression Free Interval Analysis Test Set) and evaluation of performance for individual grading algorithms (Nottingham Score Test Set). For prognostic model development, the use of the tune set was to select featurization for deep learning system output (e.g., continuous output vs. discrete integer output, sum score vs. combined histologic grade). Table 1 provides information regarding dataset usage and characteristics.

TABLE 1
Dataset usage and characteristics

| | | | |
|---|---|---|---|
| Data Usage | Train | Tune | Test |

DLS Stage 1 (patch-level)

| | | | |
|---|---|---|---|
| Data source | TTH | TTH | TCGA |
| No. of cases | 405 | 142 | 662 |
| No. of H&E slides | 520 | 148 | 685 |

DLS Stage 2 (slide-level)

| | | | | |
|---|---|---|---|---|
| Data source | TTH | TTH | MLAB | TCGA |
| No. of cases | 261 | 212 | 98 | 662 |
| No. of H&E slides | 551 | 431 | 98 | 685 |
| Case-level | 144/33 | 92/38 | 89/7 | 468/147 |
| ER* (pos/neg) | | | | |
| Slide-level MC | 368/47/ | 214/59/ | 78/10/ | 193/120/ |
| score (1/2/3) | 81 | 106 | 7 | 145 |
| Slide-level NP | 58/294/ | 19/180/ | 21/57/ | 31/235/ |
| score (1/2/3) | 146 | 189 | 16 | 237 |
| Slide-level TF | 55/87/ | 37/47/ | 13/18/ | 9/93/ |
| score (1/2/3) | 357 | 303 | 64 | 369 |

| | | |
|---|---|---|
| Data usage | Tune | Test |

Prognostic Models (Cox Regression)

| | | |
|---|---|---|
| Data Source | TTH | TCGA |
| No. of cases | 354 | 829 |
| No. of H&E slides | 829 | 878 |
| Age (min, max, median) | 25/97/58 | 26/90/58 |
| Follow-up | 398/5830/2692 | 0/8556/788 |
| min/max/median (days) | | |
| No. of events (PFI) | 60 | 93 |
| No. of censored events at 5-year | 246 | 166 |

Tumor size

| | | |
|---|---|---|
| T1 | 199 | 221 |
| T2 | 112 | 476 |
| T3 | 22 | 107 |
| T4 | 5 | 23 |

Lymph node

| | | |
|---|---|---|
| N0 | 203 | 404 |
| N1 | 65 | 261 |
| N2+ | 31 | 148 |

Metastasis

| | | |
|---|---|---|
| M0 (negative) | 1 | 808 |
| M1 (positive) | NA | 13 |

Hormone receptor status

| | | |
|---|---|---|
| ER* (pos/neg) | 275/73 | 593/177 |

Nottingham Grading System

| | | |
|---|---|---|
| MC score (1/2/3) | 213/42/94 | 247/147/172 |
| NP score (1/2/3) | 16/206/127 | 38/299/286 |
| TF score (1/2/3) | 34/61/354 | 14/127/444 |

*Not all cases have associated ER status available.
DLS: Deep Learning System; ER: Estrogen Receptor; MC: Mitotic Count; NP: Nuclear Pleomorphism; TF: Tubule Formation.

### Annotations

Pathologist annotations were performed for segmentation of invasive carcinoma as well as for all three components of the Nottingham histologic grading system (mitotic count, nuclear pleomorphism, and tubule formation). Annotations for the grading components were collected as slide-level labels as well as region-level labels for specific regions of tumor. For both slide-level and region-level annotation tasks, three board-certified pathologists from a cohort of ten pathologists were randomly assigned per slide, thus resulting in triplicate annotations per region of interest and per slide.

### Deep Learning System

Four separate deep learning models were developed: one to segment invasive carcinoma within a WSI, and three machine learning processes to predict the slide-level component score for each of the three tumor features comprising the Nottingham combined histologic grade: mitotic count, nuclear pleomorphism, and tubule formation. The invasive carcinoma model was used to provide tumor masks for the Nottingham grading models.

For providing slide-level component scores, each machine learning process was used as part of a deep learning system that consists of two stages. The first stage (“patch-level”) tiled the invasive carcinoma mask regions of the WSI into individual patches for input into a convolutional neural network (CNN), providing as output a continuous likelihood score (0-1) that each patch belongs to a given class. For mitotic figure detection, this score corresponded to the likelihood of the patch corresponding to a mitotic figure. For nuclear pleomorphism and tubule formation, the model output was a likelihood score for each of the three possible grade scores of 1-3. All stage 1 models were trained using the data summarized in Table 1 and Table 2 and utilizing ResNet50x1 pre-trained on a large natural image set. Stain normalization and color perturbation were applied and the CNN models were trained until convergence. Hyperparameter configurations including patch size and magnification were selected independently for each component model. Hyperparameters and optimal configurations for each stage 1 model are summarized in Table 3.

TABLE 2
Annotation Summary

| | | Train/Tune | Test |
|---|---|---|---|
| Mitotic | Mitoses | 3,641 | 9,678 |
| Count | Annotated | | |
| | Area reviewed | 1,979 mm² | 2,193 mm² |
| Nuclear | Grade 1 | 207k patches/53 mm² | 323k patches/83 mm² |
| Pleomorphism | Grade 2 | 3.91M patches/1,001 mm² | 4.37M patches/1,119 mm² |
| | Grade 3 | 2.30M patches/589 mm² | 3.44M patches/881 mm² |
| Tubule | Grade 1 | 380k patches/97 mm² | 226k patches/58 mm² |
| Formation | Grade 2 | 1.04M patches/265 mm² | 719k patches/184 mm² |
| | Grade 3 | 4.94M patches/1,264 mm² | 6.97M patches/1,785 mm² |

TABLE 3
Hyperparameters used for model training.

| | | Invasive | Mitotic | Nuclear | Tubule |
|---|---|---|---|---|---|
| Hyperparameters | | Carcinoma | Count | Pleomorphism | Formation |
| Network | Architecture | BiT-L (ResNet 50 × 1) | | | |
| configuration | Loss function | softmax cross-entropy | | | |
| Model Inputs | Magnification | 10x | 40x | 40x | 10x |
| | Patch size | 1024 | 128 | 1024 | 1024 |
| | Stain | Stain normalization applied using reference color | | | |
| | normalization | statistics from a fixed slide. | | | |
| Data | Orientation | Left/right mirroring and all 4 rotations | | | |
| augmentation | Brightness | tf.image.random_brightness with max_delta = 0.25 | | | |
| | Saturation | tf.image.random_saturation with lower = 0.75 and | | | |
| | | upper = 1.25 | | | |
| | Hue | tf.image.random_hue with max_delta = 0.04 | | | |
| | Contrast | tf.image.random_contrast with lower = 0.25 and | | | |
| | | upper = 1.75 | | | |
| Training | Batch size | 32 | 32 | 8 | 32 |
| | Training steps | 1M with early stopping | | | |
| Optimizer | Optimizer | RMSProp | | | |
| configuration | Decay | 0.97 | 0.95 | 0.95 | 0.97 |
| | Epsilon | 0.001 | 0.001 | 0.001 | 0.001 |
| | Momentum | 0.7 | 0.9 | 0.7 | 0.7 |
| Learning rate | Initial Rates | 3e−5 | 1e−5 | 3e−6 | 3e−4 |
| | Exponential | 0.97 | 0.95 | 0.9 | 0.95 |
| | decay | | | | |
| | Decay steps | 2,000 | 20,000 | 2,000 | 10,000 |

The second stage of each deep learning system assigned a slide-level feature score (1-3) for each feature. This was done by using the stage 1 model output to train a lightweight classifier for slide-level classification. For mitotic count, the stage I output was used to calculate mitotic density values over the invasive carcinoma region, and the mitotic density values corresponding to the 5^{th}, 25^{th}, 50^{th}, 75^{th}, and 95^{th}-percentiles for each slide were used as the input features for the stage 2 model. For nuclear pleomorphism and tubule formation, the stage 2 input feature set was the mean patch-level output (mean softmax value) for each possible score (1, 2, or 3) across the invasive carcinoma region. Based on tune set results, logistic regression was selected for the stage 2 classifier for mitotic count. For nuclear pleomorphism and tubule formation, performance of different stage 2 approaches was comparable, including logistic regression, ridge regression, and random forest. Ridge regression was selected, due to its simplicity and the ease of generating continuous component scores with this approach. All classifiers were regularized with their regularization strengths chosen via a 5-fold cross validation on the training set.

### Deep Learning System Evaluation

The performance of the deep learning system was evaluated for histologic grading at both the patch-level and the slide-level using the TCGA test set and the annotations described above. Patch-level evaluation corresponds to the stage 1 model output and utilizes the annotated regions of interest as the reference standard. For mitotic count, the patch-level reference standard corresponds to cell-sized regions identified by at least two of three pathologists as a mitotic figure. All other cell-sized regions not meeting these criteria were considered negative for the purposes of mitotic count evaluation. For nuclear pleomorphism and tubule formation, the majority vote annotation for each region of interest was assigned to all patches within that region and used as the reference standard (consistent with the approach for stage 1 training labels). The maximum probability in the model output probability map was selected to obtain the final per-patch prediction. For slide-level evaluation, the majority vote for each slide-level component score was used.

For patch-level evaluation, F1 score was calculated for mitosis detection and quadratic-weighted kappa was calculated for nuclear pleomorphism and tubule formation. For slide-level evaluation, quadratic-weighted kappa was used for all components, including inter-pathologist agreement. Table 4 illustrates the performance at the patch-level and slide-level for each component.

TABLE 4
Component Model Performance

| | Nottingham | | Result |
|---|---|---|---|
| | Components | Metric | [95% CI] |
| Patch- | Mitotic Count | F1-Score | 0.60 [0.58, 0.62] |
| level | Nuclear | Quadratic-weighted Kappa | 0.45 [0.41, 0.50] |
| | Pleomorphism | | |
| | Tubule | Quadratic-weighted Kappa | 0.70 [0.63, 0.75] |
| | Formation | | |
| Slide- | Mitotic Count | Quadratic-weighted Kappa | 0.81 [0.78, 0.84] |
| level | Nuclear | Quadratic-weighted Kappa | 0.48 [0.43, 0.53] |
| | Pleomorphism | | |
| | Tubule | Quadratic-weighted Kappa | 0.75 [0.67, 0.81] |
| | Formation | | |

For the mitosis detection model (evaluated as a detection task), the mitotic figure F1 score was 0.60 (95% CI: 0.58, 0.62). For the patch-level classification models, the quadratic Kappa was 0.45 (95% CI: 0.41, 0.50) for nuclear pleomorphism and 0.70 (95% CI: 0.63, 0.75) for tubule formation. For evaluation at the slide-level, using the majority score provided by pathologists as the reference grade, quadratic-weighted kappa was 0.81 (95% CI: 0.78, 0.84) for mitotic count, 0.48 (95% CI: 0.43, 0.53) for nuclear pleomorphism, and 0.75 (95% CI: 0.67, 0.81) for tubule formation.

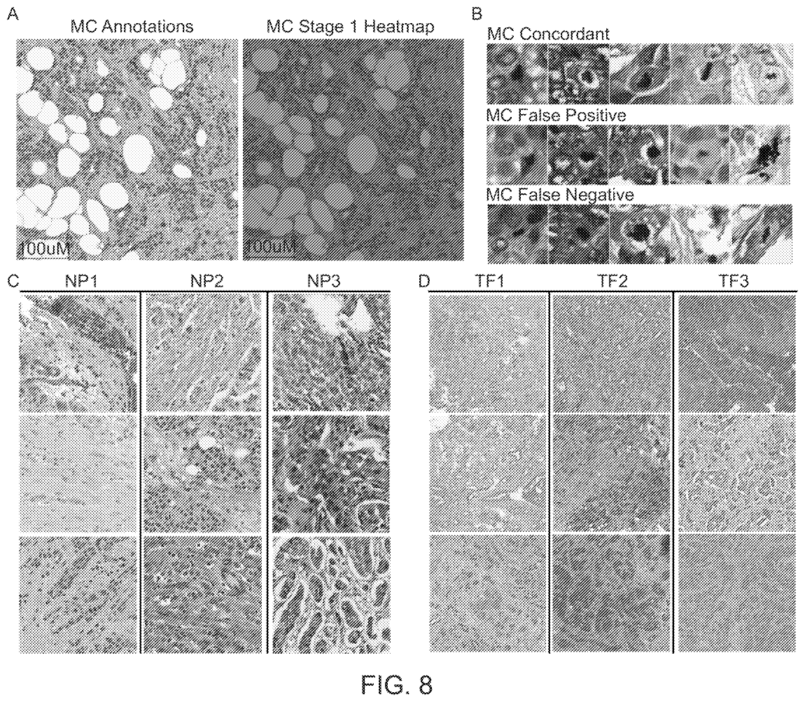

In addition, example classifications from the individual models are shown in FIG. 8. For mitotic count, pathologist annotations for mitoses corresponding to a single high-power field of 500 μm×500 μm are shown on the left and the corresponding heatmap overlay provided by the mitotic count model is shown on the right. Red regions of the overlay indicate high likelihood of a mitotic figure according to the machine learning model. Concordant patches, which correspond to regions for which pathologists and the machine learning model both identified mitotic figures, are also shown. In addition, FIG. 8 shows regions classified as mitotic figures by the machine learning model, but not identified by at least 2 of 3 reviewing pathologists (false positive), and regions identified by at least 2 of 3 reviewing pathologists as containing a mitotic figure but not classified as such by the machine learning model (false negative). Individual patches classified as grade 1, 2, 3 for nuclear pleomorphism. (40× magnification; 256 μm×256 μm) are also illustrated. Additionally, individual patches classified as grade 1, 2, or 3 for tubule formation (10× magnification; 1 mm×1 mm) are shown.

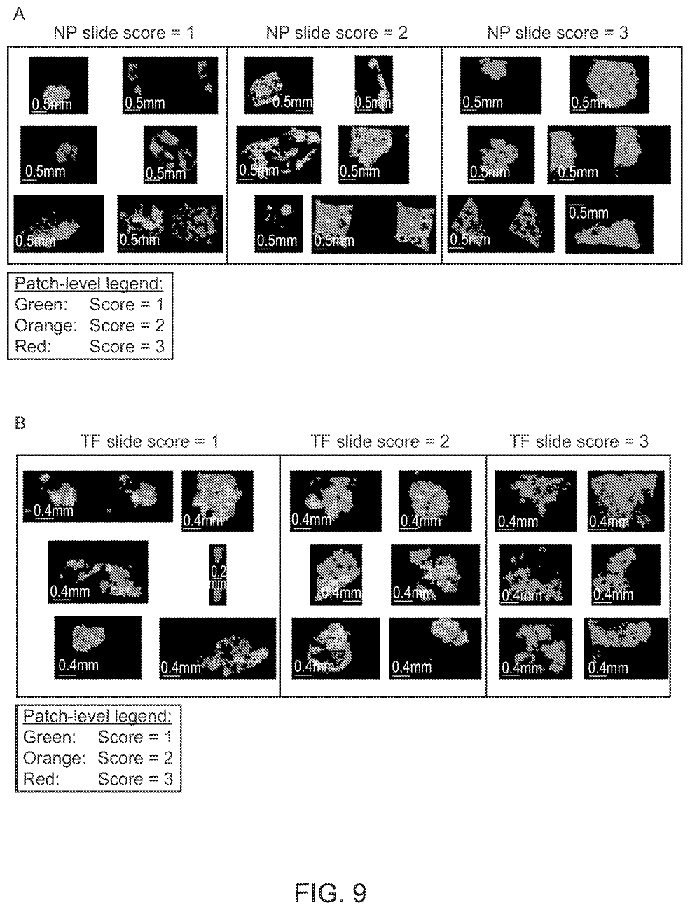

FIG. 9 illustrates examples of patch-level predictions across entire WSIs for nuclear pleomorphism and tubule formation. These were randomly sampled slides for which the slide-level score matched the majority vote pathologist slide-level score. Only regions of invasive carcinoma as identified by the invasive carcinoma model are shown. Green represents individual patches classified (argmax) with score of 1, yellow with score of 2, and red with score of 3. The patch size was 256 μm×256 μm for the nuclear pleomorphism model and 1 mm×1 mm for the tubule formation model.

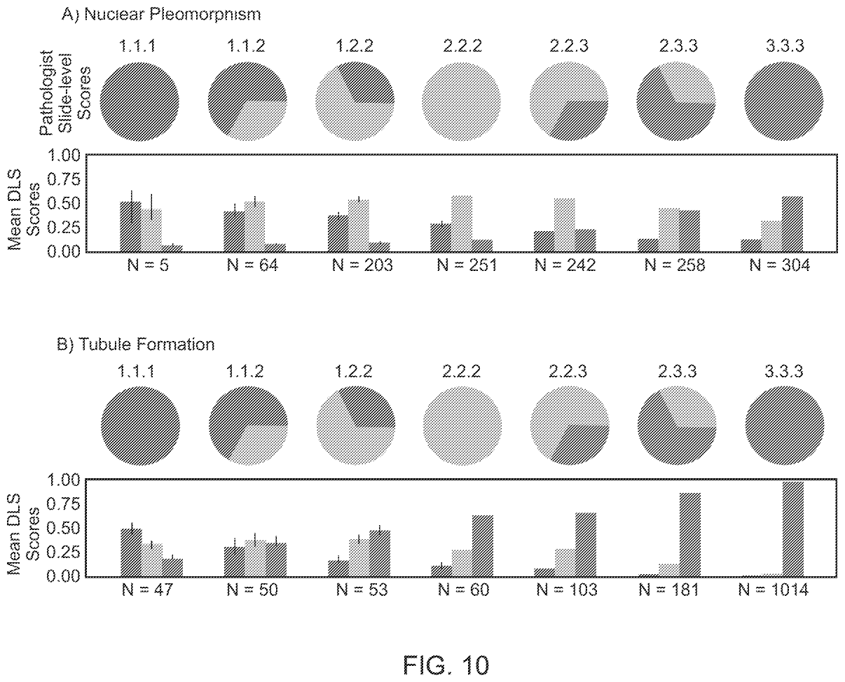

FIG. 10 illustrates an assessment of slide-level classification of nuclear pleomorphism and tubule formation by pathologists and the deep learning system. The three pathologist scores provided for each slide are represented by the pie charts. Bar plots represent the model output for each possible component score across the distribution of pathologist scores. Green corresponds to component score of 1, yellow to component score of 2, and red to component score of 3. Error bars are 95% confidence interval. The slides were grouped by the combination of pathologist scores for each slide and evaluated the deep learning system output for the resulting groups was evaluated. This analysis demonstrates that the continuous nature of the deep learning system output can reflect the distribution of pathologist agreement, whereby the output of the deep learning model produces intermediate scores for cases lacking unanimous pathologist agreement. For example, a case with a majority vote score of 1 for nuclear pleomorphism may have unanimous agreement across all three pathologists, or may have one pathologist giving a higher score, and the machine learning models were found to reflect these differences. As seen in FIG. 10, as fewer pathologists indicated a score of 1 and more pathologists indicated a score of 2 or 3, the deep learning system-estimated probability for a score of 1 (in green) decreased, and the estimated probability for a score of 3 (in red) increased.

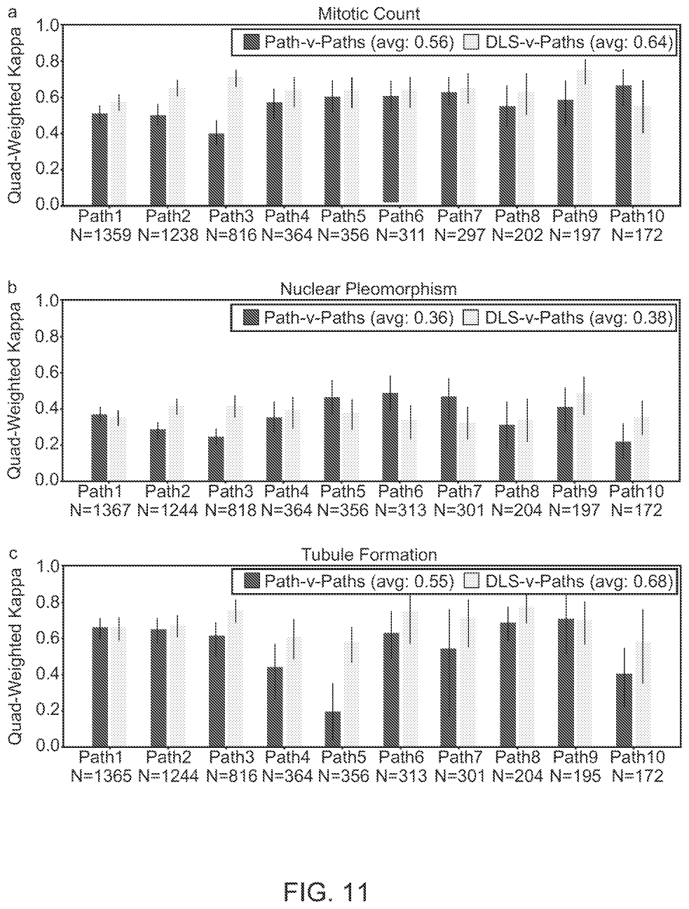

FIG. 11 illustrates inter-pathologist and deep learning system-pathologist concordance for slide-level component scoring. Each blue bar represents the agreement (quadratic-weighted kappa) between a single pathologist and the other pathologists' scores on the same cases. The yellow bar represents the agreement of the deep learning system-provided component score with all pathologists' scores on the matched set of cases. Error bars represent 95% confidence intervals computed via bootstrap. Average values in the legend represent quadratic-weighted kappa or the average of all blue bars and yellow bars, respectively. As illustrated, the average kappa (quadratic-weighted) for inter-pathologist agreement was 0.56, 0.36, 0.55 for mitotic count, nuclear pleomorphism, and tubule formation, respectively, versus 0.64, 0.38, 0.68 for the deep learning system-pathologist agreement. The kappa for inter-pathologist agreement for each individual pathologist (one vs. rest) as well for deep learning system-pathologist agreement demonstrate that, on average, the deep learning system provides consistent, pathologist-level agreement on grading of all three component features.

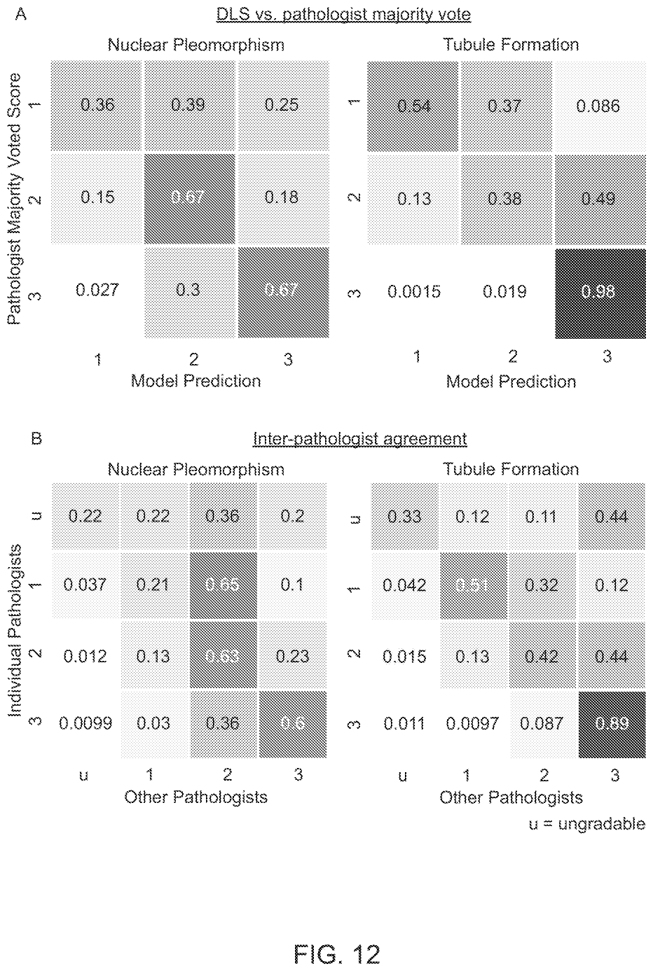

FIGS. 12-14 illustrate full confusion matrices for inter-pathologist agreement and for deep learning system agreement with the majority vote scores at the region-level, slide-level, and patch-level, respectively. In FIG. 12, deep learning system-pathologist agreement and inter-pathologist agreement for specific regions of tumor are shown. In panel A, the deep learning system output (columns) is compared to the pathologist majority vote (rows). In panel B, the pathologist scores themselves contribute to the majority vote and thus a direct comparison to panel A cannot be made. As such, the confusion matrices were calculated between each individual pathologist (rows), and the rest of pathologists that grade the same regions (columns). Then the average of these confusion matrices across all pathologists was taken to arrive at the data shown. This was done to summarize the average agreement between each pathologist and the rest of the cohort.

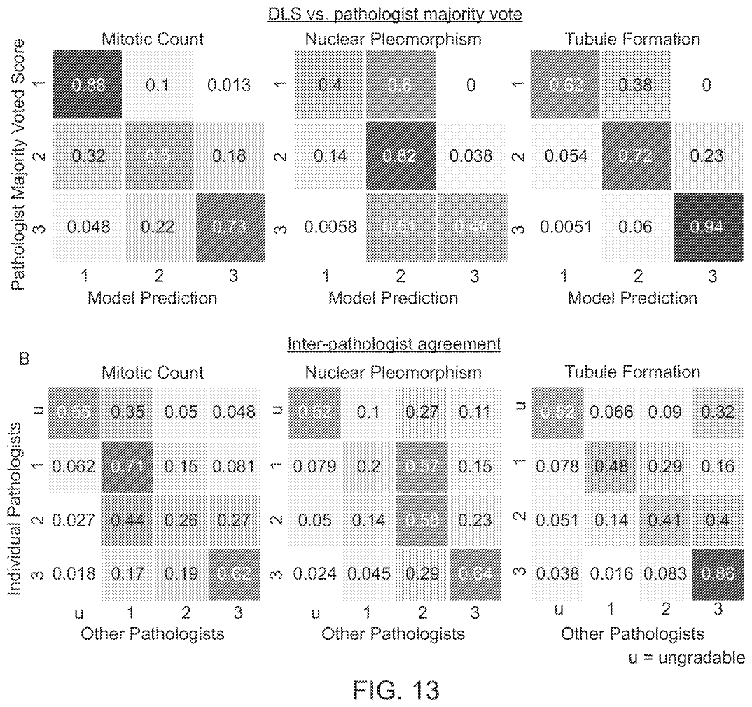

In FIG. 13, deep learning system-pathologist agreement and inter-pathologist agreement for individual whole slide images are shown. In panel A, the deep learning system output (columns) is compared to the pathologist majority vote (rows). In panel B, the confusion matrices were calculated between each individual pathologist (rows), and the rest of pathologists that grade the same regions (columns). Then the average of these confusion matrices across all pathologists was taken to arrive at the data shown. This was done to summarize the average agreement between each pathologist and the rest of the cohort.

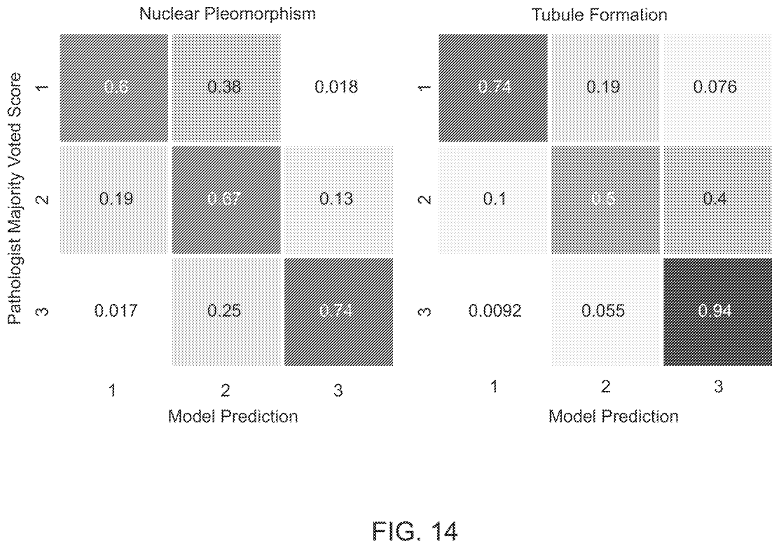

FIG. 14 illustrates patch-level deep learning system and pathologist agreement for the tune set. Model agreement with the majority vote score for individual regions are shown for nuclear pleomorphism and tubule formation, respectively. Values represent a portion of cases for each reference score with the corresponding model score.

Additional metrics to enable comparison to other studies (including unweighted kappa and precision and recall for mitotic count) are provided in Table 5 and benchmark comparisons for slide-level grading by both pathologists and computational approaches are provided in Table 6.

TABLE 5
Additional metrics for performance of component grading models

| Component models | Metric | Result [95% CI] |
|---|---|---|
| Mitotic Count | Precision | 0.76 [0.74, 0.78] |
| (patch-level) | Recall | 0.50 [0.47, 0.52] |
| Nuclear Pleomorphism | Linear-weighted Kappa | 0.42 [0.38, 0.46] |
| (patch-level) | Unweighted Kappa | 0.40 [0.35, 0.43] |
| | Accuracy | 0.67 [0.64, 0.69] |
| | Balanced Accuracy | 0.50 [0.48, 0.52] |
| Tubule Formation | Linear-weighted Kappa | 0.61 [0.55, 0.67] |
| (patch-level) | Unweighted Kappa | 0.54 [0.47, 0.59] |
| | Accuracy | 0.93 [0.91, 0.94] |
| | Balanced Accuracy | 0.67 [0.61, 0.72] |
| Mitotic Count | Linear-weighted Kappa | 0.73 [0.69, 0.77] |
| (slide-level) | Unweighted Kappa | 0.63 [0.58, 0.67] |
| Nuclear Pleomorphism | Linear-weighted Kappa | 0.40 [0.35, 0.45] |
| (slide-level) | Unweighted Kappa | 0.36 [0.30, 0.41] |
| Tubule Formation | Linear-weighted Kappa | 0.69 [0.62, 0.75] |
| (slide-level) | Unweighted Kappa | 0.64 [0.57, 0.71] |

TABLE 6
Slide-level benchmarks for grading agreement in breast cancer.

| | Kappa metric | Histologic Feature | | |
|---|---|---|---|---|
| Source | weighting | MC | NP | TF |

Inter-pathologist agreement

| | | | | |
|---|---|---|---|---|
| Rakha et al., 2017 | Unweighted | 0.34 | 0.36 | 0.49 |
| Robbins et al., 1995 | Unweighted | 0.69 | 0.46 | 0.52 |
| Frierson et al., 1995 | Unweighted | 0.52 | 0.40 | 0.64 |
| Mercan et al., 2020 | Quadratic | N/A | 0.35-0.49 | N/A |
| Ginter et al., 2021 | Fleiss | 0.28 | N/A | 0.50 |
| DLS inter-pathologist average | Quadratic | 0.56 | 0.36 | 0.55 |
| DLS-pathologist average | Quadratic | 0.64 | 0.39 | 0.69 |
| DLS-majority vote path | Quadratic | 0.81 | 0.48 | 0.75 |

Algorithm-reference standard agreement

| | | | | |
|---|---|---|---|---|
| Veta et al., 2019 | Fleiss | 0.567 | N/A | N/A |
| (Original TUPAC 16 | | | | |
| publication) | | | | |
| Mercan et al., 2020 | Quadratic | N/A | 0.43-0.56 | N/A |

### Progression Free Interval Analysis

To further evaluate the histologic grading models, the prognostic value of DLS-based grading for predicting progression free interval (PFI) was also analyzed. PFI was chosen as a clinically meaningful endpoint suitable for TCGA-BRCA. Of note, 18 cases with PFI events in the data corresponded to “new primary tumors”, predominantly of non-Breast origin according to TCGA annotations. As such, these cases were censored in the analysis, resulting in the 829 cases and 93 events summarized in Table 1.

The prognostic value of the DLS and pathologist-provided scores were evaluated both in isolation and in the context of available clinicopathologic features. As a pre-specified non-inferiority test (based on development set results), the prognostic value of the sum of continuous DLS-based component scores was compared versus the sum of discrete component scores provided by pathologists for the same images. Here, continuous scores refer to the model output corresponding to any fractional value between 1 and 3 for each component and discrete scores refer to the traditional integer scores of 1, 2, or 3. Combined histologic grade based on the summed scores (3-5: grade 1; 6-7; grade 2; 8-9 grade 3) was also evaluated in additional analyses. The prognostic performance of the two approaches is summarized in Table 7. The prognostic performance for the two approaches were similar with a c-index of 0.58 (95% CI: 0.52, 0.64) using the AI-NGS continuous sum and 0.58 (95% CI: 0.51, 0.63) using the pathologist discrete sum (Table 7; delta=0.004, lower bound of one-sided 95% CI: −0.036). This is consistent with non-inferiority of artificial intelligence-based Nottingham grading system (AI-NGS) relative to pathologist grading.

TABLE 7
Prognostic performance of direct risk prediction using
histologic scoring provided by DLS and pathologists

C-index (All cases; n = 829)

| Scoring method | DLS | Pathologist |
|---|---|---|
| Combined Histologic Grade | 0.60 | 0.58 |
| [1, 2, 3] | [0.55, 0.65] | [0.51, 0.63] |
| Summed Score Discrete | 0.59 | 0.58* |
| [3, 4, 5, 6, 7, 8, 9] | [0.53, 0.64] | [0.51, 0.63] |
| Summed Score Continuous | 0.58* | N/A |
| [3.00-9.00] | [0.52, 0.64] | |

DLS: Deep learning system.
*planned non-inferiority test comparing these configurations

While the continuous scores of the AI-NGS were utilized for primary analysis based on superior performance of this approach on the tune set, as shown in Table 8, additional approaches were also evaluated, including use of discrete summed scores (values 3-9) and the combined histologic grade (grade 1-3 based on the summed score). For pathologist grading, majority vote and originally reported diagnostic grading were also evaluated. Performance for these various scoring configurations to directly predict risk on the test set are summarized in Table 9. The c-index for AI-NGS approaches were similar, 0.58 (95% CI: 0.52, 0.64) for the AI-NGS continuous sum, 0.59 (95% CI: 0.53, 0.64) for the AI-NGS discrete sum, and 0.60 (95% CI: 0.55, 0.65) for the combined histologic grade. The pathologist-based approaches were also similar, ranging from 0.58 (95% CI: 0.51, 0.63) for pathologist combined histologic grades (1-3) to 0.61 (95% CI: 0.54, 0.66) for the majority vote summed score (3-9).

TABLE 8
Tune set prognostic performance for direct risk prediction
using histologic scoring provided by DLS and pathologists.

C-index (All cases; n = 348)

| Scoring method | DLS | Pathologist |
|---|---|---|
| Summed Score Continuous | 0.62 | N/A |
| [3.00-9.00] | [0.54, 0.69] | |
| Summed Score Discrete | 0.62 | 0.59 |
| [3, 4, 5, 6 ,7, 8, 9] | [0.54, 0.69] | [0.51, 0.66] |
| Combined Histologic Grade | 0.61 | 0.57 |
| [1, 2, 3] | [0.54, 0.68] | [0.50, 0.63] |

TABLE 9
C-index for histologic score using alternate configurations of DLS and pathologist
scoring (direct risk prediction without incorporating clinical variable).

| A) All Cases | | | |
|---|---|---|---|
| | C-index | C-index | C-index |
| Histologic | (All cases; n = 829) | (ER+ only; n = 593) | (ER+/HER2−; n = 479) |
| grade score | [95% CI] | [95% CI] | [95% CI] |

| configuration | Values | DLS | Pathologist | DLS | Pathologist | DLS | Pathologist |
|---|---|---|---|---|---|---|---|
| Summed Score | Integer | 0.59 | 0.58 | 0.61 | 0.58 | 0.62 | 0.57 |
| Discrete | in [3, 9] | [0.53, 0.64] | [0.51, 0.63]* | [0.55, 0.68] | [0.50, 0.65] | [0.55, 0.70] | [0.47, 0.66] |
| Combined | [1, 2, 3] | 0.60 | 0.58 | 0.62 | 0.58 | 0.63 | 0.57 |
| Histologic | | [0.55, 0.65] | [0.51, 0.63] | [0.56, 0.69] | [0.50, 0.65] | [0.56, 0.70] | [0.48, 0.66] |
| Grade | | | | | | | |
| Summed Score | Float | 0.58 | N/A | 0.58 | N/A | 0.57 | N/A |
| Continuous | Number | [0.52, 0.64]* | | [0.51, 0.66] | | [0.48, 0.65] | |
| | in [3, 9] | | | | | | |
| Summed Score | 0.333 | N/A | 0.61 | N/A | 0.62 | N/A | 0.63 |
| Average | increments | | [0.55, 0.66] | | [0.54, 0.69] | | [0.55, 0.71] |
| | [3, 9] | | | | | | |
| Majority-voted | Integer | N/A | 0.61 | N/A | 0.63 | N/A | 0.63 |
| Summed Score | in [3, 9] | | [0.54, 0.66] | | [0.55, 0.70] | | [0.55, 0.72] |
| Majority-voted | [1, 2, 3] | N/A | 0.60 | N/A | 0.63 | N/A | 0.62 |
| Histologic | | | [0.53, 0.65] | | [0.56, 0.70] | | [0.54, 0.74] |
| Grade | | | | | | | |

B) Restricted to cases with available pathology report scoring

| | | | | C-index (cases | | C-index (cases | |
|---|---|---|---|---|---|---|---|
| | | C-index (cases | | with path report | | with path report | |
| | | with path report | | and ER+ only; | | and ER+/HER2− | |
| Histologic grade | | only; n = 550) | | n = 426) | | only; n = 342) | |
| score | | [95% CI] | | [95% CI] | | [95% CI] | |
| configuration | Values | DLS | Pathologist | DLS | Pathologist | DLS | Pathologist |
| Summed Score | Integer | 0.60 | 0.57 | 0.63 | 0.56 | 0.64 | 0.56 |
| Discrete | in [3, 9] | [0.53, 0.67] | [0.49, 0.65] | [0.54, 0.72] | [0.46, 0.68] | [0.54, 0.75] | [0.42, 0.71] |
| Combined | [1, 2, 3] | 0.61 | 0.57 | 0.63 | 0.56 | 0.64 | 0.56 |
| Histologic Grade | | [0.54, 0.67] | [0.49, 0.64] | [0.55, 0.73] | [0.46, 0.66] | [0.54, 0.74] | [0.43, 0.69] |
| Summed Score | Float | 0.58 | | 0.57 | | 0.57 | |
| Continuous | Number | [0.50, 0.66] | N/A | [0.47, 0.68] | N/A | [0.44, 0.69] | N/A |
| | in [3, 9] | | | | | | |
| Summed Score | 0.333 | N/A | 0.61 | | 0.61 | | 0.62 |
| Average | increments | | [0.53, 0.68] | NA | [0.51, 0.71] | NA | [0.50, 0.74] |
| | [3, 9] | | | | | | |
| Majority-voted | Integer | N/A | 0.61 | | 0.61 | | 0.62 |
| Summed Score | in [3, 9] | | [0.53, 0.69] | N/A | [0.51, 0.71] | N/A | [0.50, 0.74] |
| Majority-voted | [1, 2, 3] | N/A | 0.60 | | 0.62 | | 0.61 |
| Histologic Grade | | | [0.52, 0.67] | N/A | [0.52, 0.71] | N/A | [0.49, 0.73] |
| Pathology | Integer | N/A | 0.62 | | 0.62 | | 0.62 |
| Report Summed | in [3, 9] | | [0.54, 0.70] | N/A | [0.50, 0.73] | N/A | [0.49, 0.74] |
| Score | | | | | | | |
| Pathology | [1, 2, 3] | N/A | 0.60 | | 0.60 | | 0.62 |
| Report | | | [0.52, 0.68] | N/A | [0.49, 0.71] | N/A | [0.50, 0.74] |
| Histologic Grade | | | | | | | |

B) Restricted to Cases with Available Pathology Report Scoring
To avoid comparing performance on different cases when evaluating original pathology report data, these data represent only the subset of cases for which original pathology reports were available (n=550 for all cases, and n=426 for ER+ only)

The association of each individual grading component with prognosis was also evaluated independently (Table 10). The highest discrete prognostic value for deep learning-based grading of a single feature on the test set was achieved for mitotic count, with a c-index of 0.58 (95% CI: 0.53, 0.64). The pathologist's mitotic count score gave a c-index of 0.54 (95% CI: 0.48, 0.59).

TABLE 10
Prognostic performance for using individual components of histologic grade

| | | |
|---|---|---|
| c-index (full | c-index (ER+; | c-index (ER+/ |
| dataset; n = 829) | n = 593) | HER2−; n = 479) |

DLS

| | | | |
|---|---|---|---|
| Continuous MC | 0.59 [0.53, 0.65] | 0.60 [0.52, 0.69] | 0.60 [0.49, 0.70] |
| Continuous NP | 0.55 [0.48, 0.61] | 0.54 [0.46, 0.62] | 0.51 [0.43, 0.60] |
| Continuous TF | 0.49 [0.43, 0.55] | 0.48 [0.40, 0.55] | 0.48 [0.41, 0.56] |
| Discrete MC | 0.58 [0.53, 0.64] | 0.60 [0.53, 0.68] | 0.62 [0.53, 0.70] |
| Discrete NP | 0.52 [0.47, 0.57] | 0.51 [0.46, 0.58] | 0.51 [0.45, 0.57] |
| Discrete TF | 0.54 [0.50, 0.57] | 0.54 [0.49, 0.59] | 0.54 [0.48, 0.60] |

Pathologist

| | | | |
|---|---|---|---|
| Single Pathologist MC | 0.54 [0.48, 0.59] | 0.52 [0.44, 0.60] | 0.52 [0.43, 0.62] |
| Single Pathologist NP | 0.56 [0.51, 0.61] | 0.57 [0.49, 0.64] | 0.54 [0.46, 0.62] |
| Single Pathologist TF | 0.55 [0.51, 0.58] | 0.56 [0.51, 0.60] | 0.56 [0.50, 0.62] |
| Majority voted MC | 0.58 [0.52, 0.63] | 0.59 [0.52, 0.67] | 0.61 [0.52, 0.69] |
| Majority voted NP | 0.57 [0.52, 0.63] | 0.59 [0.52, 0.66] | 0.57 [0.50, 0.65] |
| Majority voted TF | 0.55 [0.52, 0.58] | 0.55 [0.50, 0.59] | 0.56 [0.50, 0.61] |
| Average MC | 0.58 [0.52, 0.64] | 0.60 [0.52, 0.68] | 0.62 [0.53, 0.70] |
| Average NP | 0.58 [0.52, 0.64] | 0.57 [0.48, 0.65] | 0.56 [0.46, 0.65] |
| Average TF | 0.56 [0.52, 0.61] | 0.57 [0.50, 0.63] | 0.57 [0.50, 0.64] |
| Historical MC | 0.65 [0.57, 0.71] | 0.65 [0.54, 0.74] | 0.67 [0.56, 0.78] |
| Historical NP | 0.55 [0.47, 0.61] | 0.54 [0.44, 0.64] | 0.52 [0.41, 0.62] |
| Historical TF | 0.52 [0.46, 0.57] | 0.51 [0.43, 0.58] | 0.50 [0.41, 0.60] |

MC: Mitotic Count;
NP: Nuclear Pleomorphism;
TF: Tubule Formation

The prognostic value of AI-NGS in the context of established baseline variables (ER status, tumor size, nodal involvement, and age) was also evaluated. Multivariable Cox regression models were fit on the test set (TCGA-BRCA) using either the DLS-based component scores or the pathologist-provided component scores. To evaluate for improved prognostic value when adding AI-NGS information to the baseline variables, likelihood-ratio tests (LRT) were performed for cox models fit on baseline clinicopathologic variables alone versus models fit on baseline variables combined with grading scores. The corresponding c-index for the risk scores provided by these models on the test were also calculated, as reported in Table 11.

TABLE 11
Prognostic performance using summation of histologic components
in combination with baseline clinical and pathologic features.

| | | p-value (LRT) for |
|---|---|---|
| | | adding features |
| Model features | c-index | to baseline |
| Baseline Features Only | 0.74 [0.67, 0.81] | N/A (reference) |
| Baseline + AI-NGS | 0.76 [0.69, 0.81] | 0.036 |
| Baseline + Single Pathologist | 0.75 [0.69, 0.81] | 0.064 |
| Baseline + Majority Pathologist | 0.76 [0.70, 0.82] | 0.023 |

Cox models were fitted and evaluated directly on the test set and p-values are for likelihood ratio test of baseline versus baseline plus grading scores. Baseline features include age (continuous), TNM (categorical), and ER status (binary). Number of cases represents all cases with baseline characteristics available (n=762 cases; 82 events). Majority pathologist refers to the majority voted scores of three pathologists. Confidence intervals computed via bootstrap with 1000 iterations.

Overall, adding AI-NGS provided improved prognostic value over the baseline variables alone (p=0.036; likelihood ratio test of full model versus baseline model; Table 11). To better understand the potential contribution of each component feature, a similar analysis was performed using the score for each feature independently. Analysis for each component feature individually suggested improved prognostic value specifically for the AI-based mitotic count score (p=0.041; likelihood ratio test) but not the other features, as shown in Table 12. Additionally, in univariable hazard ratio (HR) analysis, the mitotic count score provided the only AI-NGS feature with a p-value less than 0.05 (HR=1.30, p=0.015), as shown in Table 13A. In multivariable analysis adjusting for ER status, tumor size (T-category), nodal involvement, and age this corresponded to a HR of 1.29 (p=0.061), as shown in Table 13B.

TABLE 12
Prognostic performance using combination of histologic components
and baseline clinical and individual component features.

| | | p-value |
|---|---|---|
| | | (Likelihood |
| | | Ratio Test) for |
| | | adding features |
| Model features | c-index | to baseline |
| Baseline Features Only | 0.74 [0.67, 0.81] | N/A (reference) |

Mitotic Count Score

| | | |
|---|---|---|
| Baseline + AI-NGS MC | 0.75 [0.69, 0.81] | 0.041 |
| Baseline + Single Pathologist MC | 0.74 [0.68, 0.81] | 0.551 |
| Baseline + Majority | 0.75 [0.68, 0.81] | 0.246 |
| Pathologist MC | | |

Nuclear Pleomorphism Score

| | | |
|---|---|---|
| Baseline + AI-NGS NP | 0.74 [0.68, 0.81] | 0.687 |
| Baseline + Single Pathologist NP | 0.75 [0.69, 0.81] | 0.125 |
| Baseline + Majority | 0.75 [0.69, 0.81] | 0.018 |
| Pathologist NP | | |

Tubule Formation Score

| | | |
|---|---|---|
| Baseline + AI-NGS TF | 0.75 [0.69, 0.81] | 0.053 |
| Baseline + Single Pathologist TF | 0.75 [0.68, 0.81] | 0.015 |
| Baseline + Majority | 0.75 [0.69, 0.81] | 0.070 |
| Pathologist TF | | |

Cox models were fit and evaluated directly on the test set and p-values are for likelihood ratio test of baseline versus baseline plus grading scores. Baseline features include age (continuous), TNM (categorical), and ER status (binary). Number of cases represents all cases with baseline characteristics available (n=762 cases; 82 events). Confidence intervals computed via bootstrap with 1000 iterations.

TABLE 13
Cox regression on the test set using pathologist
grading or AI-NGS scores and baseline variables,
in (A) univariable and (B) multivariable analysis.

| | Pathologist | | AI-NGS | |
|---|---|---|---|---|
| Feature | HR | p | HR | p |

A)

| | | | | |
|---|---|---|---|---|
| Age | 1.01 [0.99, 1.02] | 0.378 | 1.01 [0.99, 1.02] | 0.213 |

ER status

| | | | | |
|---|---|---|---|---|
| Negative | 1.0 (ref) | N/A | 1.0 (ref) | N/A |
| Positive | 0.59 [0.39, 0.91] | 0.017 | 0.60 [0.39, 0.92] | 0.018 |

Metastasis present

| | | | | |
|---|---|---|---|---|
| Negative | 1.0 (ref) | N/A | 1.0 (ref) | N/A |
| Positive | 14.03 [7.36, 26.76] | <0.001 | 14.09 [7.39, 26.88] | <0.001 |

Nodal status

| | | | | |
|---|---|---|---|---|
| N0 | 1.0 (ref) | N/A | 1.0 (ref) | N/A |
| N1+ | 2.09 [1.41, 3.09] | <0.001 | 2.10 [1.42, 3.11] | <0.001 |

Tumor Size

| | | | | |
|---|---|---|---|---|
| T1 | 1.0 (ref) | N/A | 1.0 (ref) | N/A |
| T2 | 0.89 [0.61, 1.30] | 0.559 | 0.90 [0.62, 1.30] | 0.566 |
| T3+ | 2.38 [1.56, 3.64] | <0.001 | 2.39 [1.56, 3.65] | <0.001 |
| MC | 1.27 [1.02, 1.56] | 0.028 | 1.30 [1.05, 1.61] | 0.015 |
| NP | 1.16 [0.86, 1.58] | 0.336 | 0.97 [0.69, 1.37] | 0.879 |
| TF | 1.60 [1.03, 2.50] | 0.038 | 1.35 [0.89, 2.04] | 0.157 |

B)

| | | | | |
|---|---|---|---|---|
| Age | 1.01 [0.99, 1.03] | 0.230 | 1.01 [0.99, 1.03] | 0.213 |

ER status

| | | | | |
|---|---|---|---|---|
| Negative | 1.0 (ref) | N/A | 1.0 (ref) | N/A |
| Positive | 0.54 [0.33, 0.86] | 0.010 | 0.55 [0.33, 0.89] | 0.016 |

Metastasis present

| | | | | |
|---|---|---|---|---|
| Negative | 1.0 (ref) | N/A | 1.0 (ref) | N/A |
| Positive | 11.54 [5.58, 23.87] | <0.001 | 10.31 [4.85, 21.93] | <0.001 |

Nodal status

| | | | | |
|---|---|---|---|---|
| N0 | 1.0 (ref) | N/A | 1.0 (ref) | N/A |
| N1+ | 1.65 [1.05, 2.57] | 0.029 | 1.70 [1.09, 2.66] | 0.020 |

Tumor Size

| | | | | |
|---|---|---|---|---|
| T1 | 1.0 (ref) | N/A | 1.0 (ref) | N/A |
| T2 | 0.99 [0.60, 1.64] | 0.981 | 0.96 [0.58, 1.60] | 0.880 |
| T3+ | 2.28 [1.30, 4.02] | 0.004 | 2.36 [1.33, 4.18] | 0.003 |
| MC | 1.01 [0.78, 1.29] | 0.959 | 1.29 [0.99, 1.69] | 0.061 |
| NP | 1.26 [0.87, 1.83] | 0.226 | 0.91 [0.60, 1.37] | 0.649 |
| TF | 1.79 [1.04, 3.07] | 0.034 | 1.50 [0.92, 2.45] | 0.102 |

For individual component scores (MC, NP and TF), the discrete component scores were used (values are 1, 2, or 3 for each component).

### Mitotic Count and Ki-67 Expression

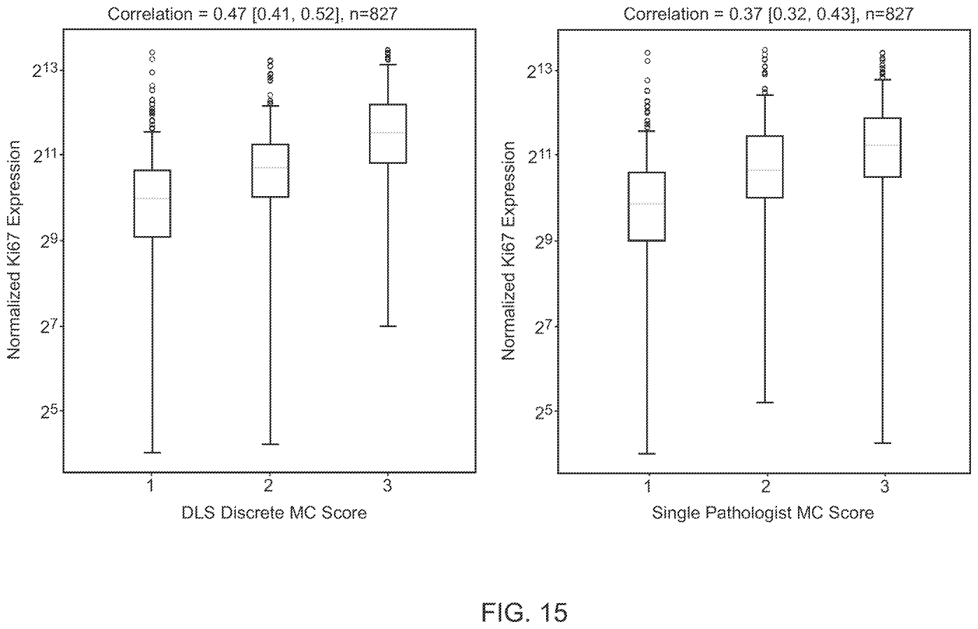

Given the potential association between mitotic count, Ki-67 (MKI67) gene expression, and prognosis, and the increasing interest of the clinical community in the use of Ki-67 in breast cancer, the correlation between the mitotic count score and Ki-67 gene expression was also evaluated. FIG. 15 illustrates the mitotic count scores provided by the deep learning system and by a pathologist. Values in brackets represent a 95% confidence interval. Box plot boxes indicate the 25th-75th percentile of Ki-67 gene expression for each mitotic count score. There is demonstrated correlation between the mitotic count score provided by the deep learning system and MKI67 expression with a correlation coefficient of 0.47 (95% CI: 0.41, 0.52) across the 827 TCGA cases with available gene expression data. For pathologist-provided mitotic count scores over the same cases, the correlation coefficient was 0.37 (95% CI: 0.32, 0.43). This indicates increased correlation with Ki-67 for the deep learning system-provided mitotic count score as compared to the mitotic count score provided by pathologist review (p=0.002 in exploratory analysis).

### Statistical Analysis

Confidence intervals were generated via bootstrap resampling with 1,000 samples. For patch-level and region-level evaluation of DLS, bootstrap resampling was performed over slides, and for progression-free interval analysis bootstrap resampling was performed over cases. All statistical tests were two-sided with the exception of the non-inferiority test, which was one-sided (with a pre-specified non-inferiority margin of 0.075 and alpha of 0.05). The margin was selected based on projected confidence intervals and power calculations using the tune dataset. No adjustment for multiple comparisons was implemented. For Ki-67 correlation analysis of mitotic count and Ki-67, permutation testing between the deep learning system and pathologist mitotic count score was performed with 1,000 samples.

### Conclusion

This systematic study characterized the performance of a deep learning system with multiple machine learning processes for components of the Nottingham grading system. The datasets were evaluated on regularization and replay methods. The machine learning processes perform both patch-level and slide-level prediction of the histologic features. The performance for each component machine learning process was shown to exceed most published benchmarks. Simultaneous development of all three machine learning processes enables a consistent, end-to-end deep learning system for Nottingham histologic grading that can also provide transparency into the underlying component features.

The automated deep learning system provides internal consistency and reliability for grading any given tumor. Such machine learning processes thus have the potential to be iteratively tuned and updated with pathologist oversight to correct error modes and stay consistent with evolving guidelines. Additionally, this study found that deep learning system-pathologist agreement generally avoids the high discordance that is sometimes observed between individual pathologists while overall trends for agreement across the three features were consistent with prior reports. Consistent, automated tools for histologic grading may help reduce discordant interpretations and mitigate the resulting complications that impact clinical care and research studies evaluating interventions, other diagnostics, and the grading systems themselves.

The continuous, consistent, and precise component scores provided by this approach also enables exploration of the individual components contributing most to the prognostic value. In the analysis, the AI-NGS provided significantly increased prognostic value relative to the baseline variables alone. Of the individual component features, mitotic count demonstrated the strongest, independent association with PFI in the analysis. Building on this, the finding that mitotic count estimation by the deep learning system correlates with Ki-67 gene expression has implications for ongoing research regarding integration of Ki-67 in prognostic models in breast cancer. Also, the stronger correlation of Ki-67 with the deep learning system mitotic count than with pathologist mitotic count suggests that for discordant mitotic figure classifications between the deep learning system and pathologists, the deep learning system may be providing more accurate representations of the biological ground truth than the pathologist-provided reference annotations.

In the test set, the summed continuous deep learning system score (floating point values in [3,9]) was not more prognostic than using a discrete, less granular, combined histologic grade (grade 1, 2, or 3). This is despite the continuous score being slightly superior on the smaller TTH “tune” data split. This may be due in part to the relatively large confidence intervals associated with the small rate of events as well as domain shifts between development and test sets due to inter-institutional differences or variability in slide processing and quality, especially given the diversity of tissue source sites in TCGA. Additionally, most TCGA cases only contributed a single slide, which may not always be most representative of the tumor and associated histologic features.

In general, this study demonstrated the potential for deep learning approaches to provide comprehensive grading in breast cancer that is on par with pathologist review. The consistent and precise nature of these models allows for potentially improved integration into prognostic models as well as enabling opportunities to efficiently evaluate correlations between morphological and molecular features.

### VIII. Additional Considerations

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

The ensuing description provides preferred exemplary embodiments only, and is not intended to limit the scope, applicability or configuration of the disclosure. Rather, the ensuing description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

1. A computer-implemented method comprising:
accessing a whole slide image of a specimen, wherein the image comprises a depiction of cells corresponding to a disease;
processing the image using a first machine learning process, wherein a first output of the first machine learning process corresponds to a mask indicating particular portions of the image predicted to depict the tumor cells;
applying the mask to the image to generate a masked image;
processing the masked image using a second machine learning process, wherein a second output of the second machine learning process corresponds to a mitotic count predicted score for a mitotic count depicted in the image;
processing the masked image using a third machine learning process, wherein a third output of the third machine learning process corresponds to a nuclear pleomorphism predicted score for nuclear pleomorphism depicted in the image;
processing the masked image using a fourth machine learning process, wherein a fourth output of the fourth machine learning process corresponds to a tubule formation predicted score for tubule formation depicted in the image;
generating a combined score of a predicted histologic grade of the disease in the image based on the second output, the third output, and the fourth output; and
outputting the combined score of the predicted histologic grade.

2. The computer-implemented method of claim 1, wherein the first machine learning process comprises a first machine learning model that segments tumor cells in the image to generate the mask.

3. The computer-implemented method of claim 1, wherein the second machine learning process comprises:
generating a first set of patches of the image, wherein each patch of the first set of patches corresponds to a portion of the image;
generating, for each patch of the first set of patches, a mitotic count patch-level score by inputting the patches into a second machine learning model, wherein the mitotic count patch-level score corresponds to a likelihood of the patch corresponding to a mitotic figure;
determining a plurality of metrics corresponding to mitotic density of the image based on the mitotic count patch-level score for each patch of the first set of patches; and
generating the mitotic count predicted score for the image by inputting the plurality of metrics into a third machine learning model.

4. The computer-implemented method of claim 1, wherein the third machine learning process comprises:
generating a second set of patches of the image, wherein each patch of the second set of patches corresponds to a portion of the image;
generating, for each patch of the second set of patches, a nuclear pleomorphism patch-level score by inputting the patch into a fourth machine learning model, wherein the nuclear pleomorphism patch-level score corresponds to a likelihood of the patch corresponding to each grade score of a plurality of grade scores associated with nuclear pleomorphism;
determining a metric associated with each grade score of the plurality of grade scores; and
generating the nuclear pleomorphism predicted score for the image by inputting the metric associated with each grade score of the plurality of grade scores into a fifth machine learning model.

5. The computer-implemented method of claim 1, wherein the fourth machine learning process comprises:
generating a third set of patches of the image, wherein each patch of the third set of patches corresponds to a portion of the image;
generating, for each patch of the third set of patches, a tubule formation patch-level score by inputting the patch into a sixth machine learning model, wherein the tubule formation patch-level score corresponds to a likelihood of the patch corresponding to each grade score of a plurality of grade scores associated with tubule formation;
determining a metric associated with each grade score of the plurality of grade scores; and
generating the tubule formation predicted score for the image by inputting the metric associated with each grade score of the plurality of grade scores into a seventh machine learning model.

6. The computer-implemented method of claim 1, wherein the first machine learning process, the second machine learning process, the third machine learning process, and the fourth machine learning process comprise a convolutional neural network.

7. The computer-implemented method of claim 1, wherein the combined score comprises a continuous score between a first value and a second value.

8. The computer-implemented method of claim 1, further comprising:
characterizing, classifying, or a combination thereof, the image with respect to the disease based on the combined score; and
outputting, an inference based on the characterizing, classifying, or the combination thereof.

9. The computer-implemented method of claim 8, further comprising: determining a diagnosis of a subject associated with the image, wherein the diagnosis is determined based on the inference.

10. The computer-implemented method of claim 9, further comprising administering a treatment to the subject based on (i) the inference and/or (ii) the diagnosis of the subject.

11. A system comprising:
one or more data processors; and
a non-transitory computer readable storage medium containing instructions which, when executed on the one or more data processors, cause the one or more data processors to perform operations comprising:
accessing a whole slide image of a specimen, wherein the image comprises a depiction of cells corresponding to a disease;
processing the image using a first machine learning process, wherein a first output of the first machine learning process corresponds to a mask indicating particular portions of the image predicted to depict the tumor cells;
applying the mask to the image to generate a masked image;
processing the masked image using a second machine learning process, wherein a second output of the second machine learning process corresponds to a mitotic count predicted score for a mitotic count depicted in the image;
processing the masked image using a third machine learning process, wherein a third output of the third machine learning process corresponds to a nuclear pleomorphism predicted score for nuclear pleomorphism depicted in the image;
processing the masked image using a fourth machine learning process, wherein a fourth output of the fourth machine learning process corresponds to a tubule formation predicted score for tubule formation depicted in the image;
generating a combined score of a predicted histologic grade of the disease in the image based on the second output, the third output, and the fourth output; and
outputting the combined score of the predicted histologic grade.

12. The system of claim 11, wherein the first machine learning process comprises a first machine learning model that segments tumor cells in the image to generate the mask.

13. The system of claim 11, wherein the second machine learning process comprises:
generating a first set of patches of the image, wherein each patch of the first set of patches corresponds to a portion of the image;
generating, for each patch of the first set of patches, a mitotic count patch-level score by inputting the patches into a second machine learning model, wherein the mitotic count patch-level score corresponds to a likelihood of the patch corresponding to a mitotic figure;
determining a plurality of metrics corresponding to mitotic density of the image based on the mitotic count patch-level score for each patch of the first set of patches; and
generating the mitotic count predicted score for the image by inputting the plurality of metrics into a third machine learning model.

14. The system of claim 11, wherein the third machine learning process comprises:
generating a second set of patches of the image, wherein each patch of the second set of patches corresponds to a portion of the image;
generating, for each patch of the second set of patches, a nuclear pleomorphism patch-level score by inputting the patch into a fourth machine learning model, wherein the nuclear pleomorphism patch-level score corresponds to a likelihood of the patch corresponding to each grade score of a plurality of grade scores associated with nuclear pleomorphism;
determining a metric associated with each grade score of the plurality of grade scores; and
generating the nuclear pleomorphism predicted score for the image by inputting the metric associated with each grade score of the plurality of grade scores into a fifth machine learning model.

15. The system of claim 1, wherein the fourth machine learning process comprises:
generating a third set of patches of the image, wherein each patch of the third set of patches corresponds to a portion of the image;
generating, for each patch of the third set of patches, a tubule formation patch-level score by inputting the patch into a sixth machine learning model, wherein the tubule formation patch-level score corresponds to a likelihood of the patch corresponding to each grade score of a plurality of grade scores associated with tubule formation;
determining a metric associated with each grade score of the plurality of grade scores; and
generating the tubule formation predicted score for the image by inputting the metric associated with each grade score of the plurality of grade scores into a seventh machine learning model.

16. The system of claim 11, wherein the first machine learning process, the second machine learning process, the third machine learning process, and the fourth machine learning process comprise a convolutional neural network.

17. The system of claim 11, wherein the combined score comprises a continuous score between a first value and a second value.

18. The system of claim 11, wherein the operations further comprise:
characterizing, classifying, or a combination thereof, the image with respect to the disease based on the combined score; and
outputting, an inference based on the characterizing, classifying, or the combination thereof.

19. The system of claim 18, wherein the operations further comprise: determining a diagnosis of a subject associated with the image, wherein the diagnosis is determined based on the inference.

20. (canceled)

21. A computer-program product tangibly embodied in a non-transitory machine-readable storage medium, including instructions configured to cause one or more data processors to perform operations comprising:
accessing a whole slide image of a specimen, wherein the image comprises a depiction of cells corresponding to a disease;
processing the image using a first machine learning process, wherein a first output of the first machine learning process corresponds to a mask indicating particular portions of the image predicted to depict the tumor cells;
applying the mask to the image to generate a masked image;
processing the masked image using a second machine learning process, wherein a second output of the second machine learning process corresponds to a mitotic count predicted score for a mitotic count depicted in the image;
processing the masked image using a third machine learning process, wherein a third output of the third machine learning process corresponds to a nuclear pleomorphism predicted score for nuclear pleomorphism depicted in the image;
processing the masked image using a fourth machine learning process, wherein a fourth output of the fourth machine learning process corresponds to a tubule formation predicted score for tubule formation depicted in the image;
generating a combined score of a predicted histologic grade of the disease in the image based on the second output, the third output, and the fourth output; and
outputting the combined score of the predicted histologic grade.

22.-30. (canceled)
